(19) United States
(12) Patent Application Publication
(10) Pub. No.: US 2007/0191431 A1
(43) Pub. Date: Aug. 16, 2007

(54) 2-Amino-5-piperidinylimidazolone compounds and use thereof for beta-secretase modulation

(75) Inventors: Ping Zhou, Plainsboro, NJ (US); Ronald Charles Bernotas, Royersford, PA (US); Yanfang Li, Lawrenceville, NJ (US); Pawel Wojciech Nowak, Montvale, NJ (US); Derek Cecil Cole, New City, NY (US); Eric Steven Manas, Lafayette Hill, PA (US); Yi Fan, Robbinsville, NJ (US); Yinfa Yan, Bedminster, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/640,751

(22) Filed: Dec. 18, 2006

Related U.S. Application Data
(60) Provisional application No. 60/751,678, filed on Dec. 19, 2005.

(51) Int. Cl. A61K 31/454; C07D 403/02; C07D 405/14

(52) U.S. Cl. 514/320; 514/326; 546/209

(57) ABSTRACT

The present invention provides a 2-amino-5-piperidinylimidazolone compound of formula I 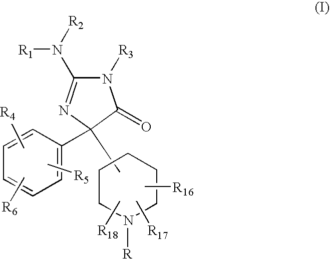 The present invention also provides methods and compositions for the inhibition of β-secretase (BACE) and the treatment of β-amyloid deposits and neurofibrillary tangles.

This application claims the benefit under 35 U.S.C. §119(e) to co-pending U.S. provisional application No. 60/751678, filed Dec. 19, 2005, which is hereby incorporated by reference in its entirety.

### BACKGROUND OF THE INVENTION

β-Amyloid deposits and neurofibrillary tangles are two major pathologic characterizations associated with Alzheimer's disease (AD). Clinically, AD is characterized by the of loss of memory, cognition, reasoning, judgment, and orientation. Also affected, as the disease progresses, are motor, sensory, and linguistic abilities until global impairment of multiple cognitive functions occurs. These cognitive losses take place gradually, but typically lead to severe impairment and eventual death in 4-12 years.

Amyloidogenic plaques and vascular amyloid angiopathy also characterize the brains of patients with Trisomy 21 (Down's Syndrome), Hereditary Cerebral Hemorrhage with Amyloidosis of the Dutch-type (HCHWA-D), and other neurodegenerative disorders. Neurofibrillary tangles also occur in other neurodegenerative disorders including dementia-inducing disorders (Varghese, J., et al, Journal of Medicinal Chemistry, 2003, 46, 4625-4630).

β-Amyloid deposits are predominately an aggregate of Aβ peptide, which in turn is a product of the proteolysis of amyloid precursor protein (APP). More specifically, Aβ peptide results from the cleavage of APP at the C-terminus by one or more γ-secretases, and at the N-terminus by β-secretase enzyme (BACE), also known as aspartyl protease, as part of the β-amyloidogenic pathway.

BACE activity is correlated directly to the generation of Aβ peptide from APP (Sinha, et al, Nature, 1999, 402, 537-540), and studies indicate that the inhibition of BACE inhibits the production of Aβ peptide (Roberds, S. L., et al, Human Molecular Genetics, 2001, 10, 1317-1324).

Therefore, it is an object of this invention to provide compounds which are inhibitors of β-secretase and are useful as therapeutic agents in the treatment, prevention or amelioration of a disease or disorder characterized by elevated β-amyloid deposits or β-amyloid levels in a patient.

It is another object of this invention to provide therapeutic methods and pharmaceutical compositions useful for the treatment, prevention or amelioration of a disease or disorder characterized by elevated β-amyloid deposits or β-amyloid levels in a patient.

It is a feature of this invention that the compounds provided may also be useful to further study and elucidate the activity of the β-secretase enzyme.

These and other objects and features of the invention will become more apparent by the detailed description set forth hereinbelow.

### SUMMARY OF THE INVENTION

The present invention provides a compound of formula I 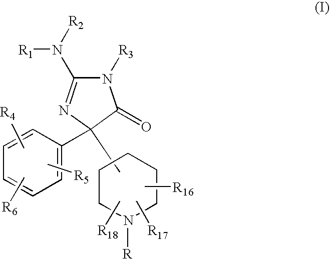
wherein
R is H, COR₇, CO₂R₇, CONR₈R₉, SO₂NR₈R₉, SOₘR₁₀, or an alkyl, alkenyl, alkynyl, cycloalkyl, cycloheteroalkyl, aryl or heteroaryl group each optionally substituted;
R₁, R₂, and R₃ are each independently H or an alkyl, cycloalkyl, cycloheteroalkyl, aryl or heteroaryl group each optionally substituted or R₁ and R₂ may be taken together with the atom to which they are attached form an optionally substituted 5- to 7-membered ring optionally interrupted by an additional heteroatom selected from O, N or S;
R₄, R₅, and R₆ are each independently H, halogen, NO₂, CN, OR₁₁, COR₁₁, CO₂R₁₁, CONR₁₂R₁₃, NR₁₂R₁₃, NR₁₂COR₁₄, NR₁₂SO₂R₁₄, SO₂NR₁₂R₁₃, SOₙR₁₄ or an alkyl, alkenyl, alkynyl, cycloalkyl, cycloheteroalkyl, aryl or heteroaryl group each optionally substituted or when attached to adjacent carbon atoms R₄ and R₅ or R₅ and R₆ may be taken together with the atoms to which they are attached to form an optionally substituted 5- to7-membered ring optionally interrupted by one, two or three heteroatoms selected from O, N or S;
m and n are each independently 0, 1 or 2;
R₇ and R₁₁, are each independently H or an alkyl, alkenyl, alkynyl, cycloalkyl, cycloheteroalkyl, aryl or heteroaryl group each optionally substituted;
R₈, R₉, R₁₂ and R₁₃ are each independently H, OR₁₅, COR₁₅, CO₂R₁₅ or an alkyl, alkenyl, alkynyl, cycloalkyl, cycloheteroalkyl, aryl or heteroaryl group each optionally substituted or R₈ and R₉ or R₁₂ and R₁₃ may be taken together with the atom to which they are attached to form an optionally substituted 5- to 7-membered ring optionally interrupted by an additional heteroatom selected from O, N or S;
R₁₀ and R₁₄ are each independently an alkyl, alkenyl, alkynyl, cycloalkyl, cycloheteroalkyl, aryl or heteroaryl group each optionally substituted;
R₁₅ is H or an alkyl, alkenyl, alkynyl, cycloalkyl, cycloheteroalkyl, aryl or heteroaryl group each optionally substituted;
R₁₆, R₁₇ and R₁₈ are each independently H, halogen, CN, OR₁₉ or an alkyl, cycloalkyl, cyclohetereoalkyl, aryl or heteroaryl group each optionally substituted; and
R₁₉ is H or an alkyl, cycloalkyl, cyclohetereoalkyl, aryl or heteroaryl group each optionally substituted; or
   a tautomer thereof, a stereoisomer thereof or a pharmaceutically acceptable salt thereof.

The present invention also relates to the use of 2-amino-5-piperidinylimidazolone compounds for the treatment of β-amyloid deposits and neurofibrillary tangles. These compounds are particularly useful in treating Alzheimer's disease, cognitive impairment, Down's Syndrome, HCHWA-D, cognitive decline, senile dementia, cerebral amyloid angiopathy, degenerative dementia, or other neurodegenerative disorders.

### DETAILED DESCRIPTION OF THE INVENTION

Alzheimer's disease (AD) is a major degenerative disease of the brain which presents clinically by progressive loss of memory, cognition, reasoning, judgement and emotional stability and gradually leads to profound mental deterioration and death. The exact cause of AD is unknown, but increasing evidence indicates that amyloid beta peptide (A-beta) plays a central role in the pathogenesis of the disease. (D. B. Schenk; R. E. Rydel et al, Journal of Medicinal Chemistry, 1995, 21, 4141 and D. J. Selkoe, Physiology Review, 2001, 81, 741). Patients with AD exhibit characteristic neuropathological markers such as neuritic plaques (and in β-amyloid angiopathy, deposits in cerebral blood vessels) as well as neurofibrillary tangles detected in the brain at autopsy. A-beta is a major component of neuritic plaques in AD brains. In addition, β-amyloid deposits and vascular β-amyloid angiopathy also characterize individuals with Downs Syndrome, Hereditary Cerebral Hemmorhage with Amyloidosis of the Dutch type and other neurodegenreative and dementia-inducing disorders. Over expression of the amyloid precursor protein (APP), altered cleavage of APP to A-beta or a decrease in the clearance of A-beta from a patient's brain may increase the levels of soluble or fibrullar forms of A-beta in the brain. The β-site APP cleaving enzyme, BACE1, also called memapsin-2 or Asp-2, was identified in 1999 (R. Vassar, B. D. Bennett, et al, Nature, 1999, 402, 537). BACE1 is a membrane-bound aspartic protease with all the known functional properties and characteristics of β-secretase. Low molecular weight, non-peptide, non-substrate-related inhibitors of BACE1 or β-secretase are earnestly sought both as an aid in the study of the β-secretase enzyme and as potential therapeutic agents.

Surprisingly, it has now been found that 2-amino-5-piperidinylimidazolone compounds of formula I demonstrate inhibition of β-secretase and the selective inhibition of BACE1. Advantageously, said piperidinylimidazolone compounds may be used as effective therapeutic agents for the treatment, prevention or amelioration of a disease or disorder characterized by elevated β-amyloid deposits or β-amyloid levels in a patient. Accordingly, the present invention provides a 2-amino-5-piperidinylimidazolone compound of formula I 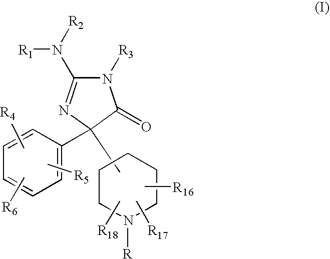
wherein
R is H, COR₇, CO₂R₇, CONR₈R₉, SO₂NR₈R₉, SOₘR₁₀, or an alkyl, alkenyl, alkynyl, cycloalkyl, cycloheteroalkyl, aryl or heteroaryl group each optionally substituted;
R₁, R₂, and R₃ are each independently H or an alkyl, cycloalkyl, cycloheteroalkyl, aryl or heteroaryl group each optionally substituted or R₁ and R₂ may be taken together with the atom to which they are attached form an optionally substituted 5- to 7-membered ring optionally interrupted by an additional heteroatom selected from O, N or S;
R₄, R₅, and R6 are each independently H, halogen, NO₂, CN, OR₁₁, COR₁₁, CO₂R₁₁, CONR₁₂R₁₃, NR₁₂R₁₃, NR₁₂COR₁₄, NR₁₂SO₂R₁₄, SO₂NR₁₂R₁₃, SOₙR₁₄ or an alkyl, alkenyl, alkynyl, cycloalkyl, cycloheteroalkyl, aryl or heteroaryl group each optionally substituted or when attached to adjacent carbon atoms R₄ and R₅ or R₅ and R₆ may be taken together with the atoms to which they are attached to form an optionally substituted 5- to7-membered ring optionally interrupted by one, two or three heteroatoms selected from O, N or S;
m and n are each independently 0, 1 or 2;
R₇ and R₁₁ are each independently H or an alkyl, alkenyl, alkynyl, cycloalkyl, cycloheteroalkyl, aryl or heteroaryl group each optionally substituted;
R₈, R₉, R₁₂ and R₁₃ are each independently H, OR₁₅, COR₁₅, CO₂R1₅ or an alkyl, alkenyl, alkynyl, cycloalkyl, cycloheteroalkyl, aryl or heteroaryl group each optionally substituted or R₈ and R₉ or R₁₂ and R₁₃ may be taken together with the atom to which they are attached to form an optionally substituted 5- to 7-membered ring optionally interrupted by an additional heteroatom selected from O, N or S;
R₁₀ and R₁₄ are each independently an alkyl, alkenyl, alkynyl, cycloalkyl, cycloheteroalkyl, aryl or heteroaryl group each optionally substituted;
R₁₅ is H or an alkyl, alkenyl, alkynyl, cycloalkyl, cycloheteroalkyl, aryl or heteroaryl group each optionally substituted;
R₁₆, R₁₇ and R₁₈ are each independently H, halogen, CN, OR₁₉ or an alkyl, cycloalkyl, cyclohetereoalkyl, aryl or heteroaryl group each optionally substituted; and
R₁₉ is H or an alkyl, cycloalkyl, cyclohetereoalkyl, aryl or heteroaryl group each optionally substituted; or
   a tautomer thereof, a stereoisomer thereof or a pharmaceutically acceptable salt thereof.

In one embodiment, R₅ is an optionally substituted heteroaryl group. Representative heteroaryl groups include pyridine, thiophene, thiazole, thiadiazole, furan, oxazole, oxadiazole, pyrrole, pyrazole, imidazole, triazole, oxathiole, isoxazole, oxazole, oxatriazole, dioxazole, oxathiazole, tetrazole, pyridazine, pyrimidine, pyrazine, triazine, oxazine, oxathiazine, or oxadiazine. The heteroaryl group may be unsubstituted or substituted with alkyl, alkoxy, trifluoroalkyl, trifluoroalkoxy, amino, halogen, hydroxyl, or CN, or forms an N-oxide. For example R₅ may be an optionally substituted pyridine or pyrimidine group.

In another embodiment, R₅ is a phenyl group optionally substituted with CN, OCF₃ or halogen.

As used herein, the term “alkyl” includes both straight chain and branched-chain (unless defined otherwise) monovalent saturated hydrocarbon moieties of 1-12 carbon atoms, preferably 1-6 carbon atoms, more preferably ‘lower’ alkyl of 1-4 carbon atoms. Examples of saturated hydrocarbon alkyl moieties include, but are not limited to, chemical groups such as methyl, ethyl, n-propyl, isopropyl, n-butyl, tert-butyl, isobutyl, sec-butyl; higher homologs such as n-pentyl, n-hexyl, and the like. Alkyl groups can be optionally substituted. Suitable alkyl substitutions include, but are not limited to, CN, OH, halogen, alkenyl, alkynyl, cycloalkyl, phenyl, carbamoyl, carbonyl, alkoxy or aryloxy.

The term “haloalkyl” as used herein designates a C,H₂ₙ₊₁ group having from one to 2n+1 halogen atoms which may be the same or different and the term haloalkoxy as used herein designates an OCₙH₂ₙ₊₁ group having from one to 2n+1 halogen atoms which may be the same or different. Preferably the term haloalkyl designates CF₃ and the term haloalkoxy designates OCF₃.

The term “alkenyl”, as used herein, refers to either a straight chain or branched-chain hydrocarbon moiety containing at least one double bond and having from 2-12 carbon atoms, preferably 2-6 carbon atoms, more preferably 2-4 carbon atoms. Such hydrocarbon alkenyl moieties may be mono or polyunsaturated, and may exist in the E or Z configurations. The compounds of this invention are meant to include all possible E and Z configurations. Examples of mono or polyunsaturated hydrocarbon alkenyl moieties include, but are not limited to, chemical groups such as vinyl, 2-propenyl, isopropenyl, crotyl, 2-isopentenyl, butadienyl, 2-(butadienyl), 2,4-pentadienyl, 3-(1,4-pentadienyl), and higher homologs, isomers, or the like.

The term “alkynyl”, as used herein, refers to an alkyl group having one or more triple carbon-carbon bonds. Alkynyl groups preferably contain 2 to 6 carbon atoms. Examples of alkynyl groups include, but are not limited to, ethynyl, propynyl, butynyl, pentynyl, and the like. In some embodiments, alkynyl groups can be substituted with up to four substituent groups, as described hereinabove.

The term “cycloalkyl”, as used herein, refers to a monocyclic, bicyclic, tricyclic, fused, bridged, or spiro saturated carbocyclic moiety of 3-10 carbon atoms. Any suitable ring position of the cycloalkyl moiety may be covalently linked to the defined chemical structure. Examples of cycloalkyl moieties include, but are not limited to, chemical groups such as cyclopropyl, cyclobutyl, cyclopentyl, cyclohexyl, cycloheptyl, norbornyl, adamantyl, spiro[4.5]decanyl, and homologs, isomers, or the like.

The term “cycloheteroalkyl”, as used herein, designates a five- to seven-membered cycloalkyl ring system containing 1 or 2 heteroatoms, which may be the same or different, selected from N, O or S and optionally containing one double bond. Exemplary of the cycloheteroalkyl ring systems included in the term as designated herein are the following rings wherein X is NR′, 0 or S; and R′ is H or an optional substituent as described hereinbelow:

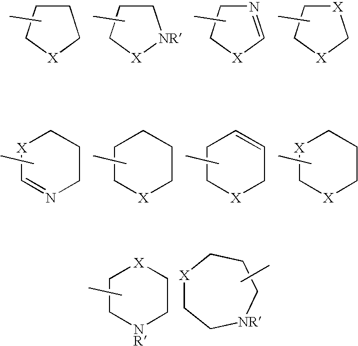

The term “aryl”, as used herein, designates an aromatic carbocyclic moiety of up to 20 carbon atoms, e.g. 6-20 carbon atoms, which may be a single ring (monocyclic) or multiple rings (bicyclic, up to three rings) fused together or linked covalently. Examples of aryl moieties include, but are not limited to, chemical groups such as phenyl, 1-naphthyl, 2-naphthyl, dihydronaphthyl, tetrahydronaphthyl, biphenyl, anthryl, phenanthryl, fluorenyl, indanyl, biphenylenyl, acenaphthenyl, acenaphthylenyl, and the like. In some embodiments “aryl” groups can be substituted with from 1-5 substituents.

The term “heteroaryl” as used herein designates an aromatic heterocyclic ring system, e.g. having from 5-20 ring atoms, which may be a single ring (monocyclic) or multiple rings (bicyclic, up to three rings) fused together or linked covalently. Preferably, heteroaryl is a 5- to 6-membered ring. The rings may contain from one to four hetero atoms selected from nitrogen, oxygen, or sulfur, wherein the nitrogen or sulfur atom(s) are optionally oxidized, or the nitrogen atom(s) are optionally quaternized. Examples of heteroaryl moieties include, but are not limited to, heterocycles such as furan, thiophene, pyrrole, pyrazole, imidazole, oxazole, isoxazole, thiazole, isothiazole, 1H-tetrazole, 1,3,4-oxadiazole, 1H-1,2,4-triazole, 1,3,4-triazole, pyridine, pyrimidine, pyrazine, pyridazine, benzoxazole, benzisoxazole, benzothiazole, benzofuran, benzothiophene, thianthrene, benzimidazole, indole, indazole, quinoline, isoquinoline, quinazoline, quinoxaline, purine, pteridine, 9H-carbazole, α-carboline, or the like.

The term “halogen”, as used herein, designates fluorine, chlorine, bromine, or iodine.

In the specification and claims, when the terms alkyl, alkenyl, alkynyl, cycloalkyl, cycloheteroalkyl, aryl or heteroaryl are designated as being optionally substituted, the substituent groups which are optionally present may be one or more of those customarily employed in the development of pharmaceutical compounds or the modification of such compounds to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, carboxyalkoxy, carboxyalkyl, alkanoyl, alkylthio, alkylsuphinyl, alkylsulphonyl, carbamoyl, alkylamido, phenyl, phenoxy, benzyl, benzyloxy, heterocyclyl or cycloalkyl groups, preferably halogen atoms or lower alkyl or lower alkoxy groups. Typically, 0-3 substituents may be present. When any of the foregoing substituents represents or contains an alkyl substituent group, this may be linear or branched and may contain up to 12, preferably up to 6, more preferably up to 4 carbon atoms.

Pharmaceutically acceptable salts may be any acid addition salt formed by a compound of formula I and a pharmaceutically acceptable acid such as phosphoric, sulfuric, hydrochloric, hydrobromic, citric, maleic, malonic, mandelic, succinic, fumaric, acetic, lactic, nitric, sulfonic, p-toluene sulfonic, methane sulfonic acid or the like. Where the compound of formula I contains an acidic function such as a carboxyl group, the pharmaceutically acceptable salts may be derived from a base, for instance a sodium salt.

Compounds of the invention include esters, carbamates or other conventional prodrug forms, which in general, are functional derivatives of the compounds of the invention and which are readily converted to the inventive active moiety in vivo. Correspondingly, the method of the invention embraces the treatment of the various conditions described hereinabove with a compound of formula I or with a compound which is not specifically disclosed but which, upon administration, converts to a compound of formula I in vivo. Also included are metabolites of the compounds of the present invention defined as active species produced upon introduction of these compounds into a biological system.

Compounds of the invention may exist as one or more tautomers. One skilled in the art will recognize that compounds of formula I may also exist as the tautomer (It) as shown below.

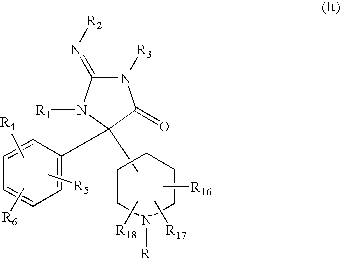

Tautomers often exist in equilibrium with each other. As these tautomers interconvert under environmental and physiological conditions, they provide the same useful biological effects. The present invention includes mixtures of such tautomers as well as the individual tautomers of Formula I and Formula It.

The compounds of this invention may contain an asymmetric carbon atom and some of the compounds of this invention may contain one or more asymmetric centers and may thus give rise to optical isomers and diastereomers. While shown without respect to stereochemistry in Formula I, the present invention includes such optical isomers and diastereomers; as well as the racemic and resolved, enantiomerically pure R and S stereoisomers; as well as other mixtures of the R and S stereoisomers and pharmaceutically acceptable salts thereof. Where a stereoisomer is preferred, it may in some embodiments be provided substantially free of the corresponding enantiomer. Thus, an enantiomer substantially free of the corresponding enantiomer refers to a compound that is isolated or separated via separation techniques or prepared free of the corresponding enantiomer. “Substantially free”, as used herein, means that the compound is made up of a significantly greater proportion of one stereoisomer, preferably less than about 50%, more preferably less than about 75%, and even more preferably less than about 90%.

Preferred compounds of the invention are those compounds of formula I wherein R₁₆, R₁₇ and R₁₈ are H. Another group of preferred compounds are those compounds of formula I wherein R₆ is NR₁₂COR₁₄ or an optionally substituted aryl or heteroaryl group. A further group of preferred compounds are those formula I compounds wherein R₃ is alkyl.

More preferred compounds of the invention are those compounds of formula I wherein the piperidinyl ring is attached in the 3- or 4-position. Another group of more preferred compounds is those compounds of formula I wherein the piperidinyl ring is attached in the 3- or 4-position; R is COR₇; and R₁ and R₂ are H. A further group of more preferred compounds are those compounds of formula I wherein the piperidinyl ring is attached in the 3- or 4-position; R is COR₇; R₆ is NR₁₂COR₁₄ or an optionally substituted phenyl or heteroaryl group; and R₁₆, R₁₇ and R₁₈ are H.

Preferred compounds of the invention include:
2-amino-5-(1,1′-biphenyl-3-yl)-5-(1-isobutyrylpiperidin-4-yl)-3-methyl-3,5-dihydro-4H -imidazol-4-one;
2-amino-5-(1-benzoylpiperidin-4-yl)-5-(1,1′-biphenyl-3-yl)-3-methyl-3,5-dihydro -4H -imidazol-4-one;
2-amino-5-(1,1′-biphenyl-3-yl)-5-[1-(3-methoxybenzoyl)piperidin-4-yl]-3-methyl-3,5-dihydro-4H-imidazol-4-one;
2-amino-5-(1,1′-biphenyl-3-yl)-5-[1-(2-furoyl)piperidin-4-yl]-3-methyl -3,5-dihydro-4H -imidazol-4-one;
2-amino-5-(1,1′-biphenyl-3-yl)-5-[1-(2-methoxybenzoyl)piperidin-4-yl]-3-methyl-3,5-dihydro-4H-imidazol-4-one;
2-amino-5-(1,1′-biphenyl-3-yl)-5-[1-(4-methoxybenzoyl)piperidin-4-yl]-3-methyl-3,5-dihydro4H-imidazol-4-one;
2-amino-5-(1,1′-biphenyl-3-yl)-5-[1-(3,4-dimethoxybenzoyl)piperidin-4-yl]-3-methyl -3,5-dihydro4H-imidazol-4-one;
2-amino-5-[1-(1,3-benzodioxol-5-ylcarbonyl)piperidin-4-yl]-5-(1,1′-biphenyl-3-yl)-3-methyl-3,5-dihydro4H-imidazol-4-one;
2-amino-5-(1, 1′-biphenyl-3-yl)-3-methyl-5-[1-(1-naphthoyl)piperidin-4-yl]-3,5-dihydro -4H-imidazol-4-one;
2-amino-5-(1,1′-biphenyl-3-yl)-3-methyl-5-[1-(4-propylbenzoyl)piperidin4-yl]-3,5-dihydro4H-imidazol-4-one;
2-amino-5-(1,1′-biphenyl-3-yl)-3-methyl-5-[1-(4-propoxybenzoyl)piperidin4-yl]-3,5-dihydro4H-imidazol-4-one;
2-({4-[2-amino4-(1,1′-biphenyl-3-yl)-1-methyl-5-oxo-4,5-dihydro-1H -imidazol-4-yl]piperidin-1-yl}carbonyl)benzonitrile;
3-({4-[2-amino-4-(1,1′-biphenyl-3-yl)-1-methyl-5-oxo-4,5-dihydro-1H -imidazol-4-yl]piperidin-1-yl}carbonyl)benzonitrile;
4-({4-[2-amino-4-(1,1′-biphenyl-3-yl)-1-methyl-5-oxo-4,5-dihyd ro-1H -imidazol-4-yl]piperidin-1-yl}carbonyl)benzonitrile;
2-amino-5-(1,1′-biphenyl-3-yl)-5-[1-(2-chloro-6-methylisonicotinoyl)piperidin-4-yl]-3-methyl-3,5-dihydro-4H-imidazol-4-one;
2-amino-5-(1,1′-biphenyl-3-yl)-5-[1-(3-furoyl)piperidin-4-yl]-3-methyl -3,5-dihydro-4H -imidazol-4-one;
2-amino-5-(1,1′-biphenyl-3-yl)-3-methyl-5-[1-(thien-2-ylcarbonyl)piperidin-4-yl]-3,5-dihydro-4H-imidazol-4-one;
2-amino-5-(1,1′-biphenyl-3-yl)-3-methyl-5-[1-(thien-3-ylcarbonyl)piperidin-4-yl]-3,5-dihydro-4H-imidazol-4-one;
2-amino-5-(1,1′-biphenyl-3-yl)-3-methyl-5-[1-(phenylsulfonyl)piperidin-4-yl]-3,5-dihydro-4H-imidazol-4-one;
2-amino-5-{1-[(benzyloxy)acetyl]piperidin4-yl}-5-(1,1′-biphenyl-3-yl)-3-methyl-3,5-dihydro-4H-imidazol-4-one;
2-amino-5-(1,1′-biphenyl-3-yl)-3-methyl-5-(1-prop-2-ynylpiperidin4-yl) -3,5-dihydro -4H-imidazol-4-one;
5-(1-acetylpiperidin4-yl)-2-amino-5-(1,1′-biphenyl-3-yl)-3-methyl-3,5-dihydro -4H -imidazol-4-one;
2-amino-5-(1,1′-biphenyl-3-yl)-3-methyl-5-(1-propionylpiperidin-4-yl)-3,5-dihydro -4H -imidazol-4-one;
2-amino-5-(1,1′-biphenyl-3-yl)-5-(1-butyrylpiperidin-4-yl)-3-methyl-3,5-dihydro -4H -imidazol-4-one
2-amino-5-(1-benzoylpiperidin-4-yl)-5-(3-cyclohexylphenyl)-3-methyl-3,5-dihydr-4H -imidazol-4-one;
5-(1-acetylpiperidin-4-yl)-2-amino-5-(3-cyclohexylphenyl)-3-methyl-3,5-dihydr-4H -imidazol-4-one;
2-amino-5-(1-benzoylpiperidin-4-yl)-3-methyl-5-(3-pyridin-3-ylphenyl)-3,5-dihydro-4H -imidazol-4-one;
2-amino-5-(1-benzoylpiperidin-4-yl)-3-methyl-5-(3-pyrimidin-5-ylphenyl)-3,5-dihydro-4H-imidazol-4-one;
2-amino-5-(1-benzoylpiperidin-4-yl)-3-methyl-5-(3-pyrazin-2-ylphenyl)-3,5-dihydro -4H-imidazol-4-one;
2-amino-5-(1-benzoylpiperidin-4-yl)-5-(2′,5′-difluoro-1,1′-biphenyl-3-yl)-3-methyl-3,5-dihydro-4H-imidazol-4-one;
2-amino-5-(1-benzoylpiperidin-4-yl)-3-methyl-5-(3-propoxyphenyl)-3,5-dihydro-4H -imidazol-4-one;
2-amino-5-(1-benzoylpiperidin-4-yl)-5-(3-isobutoxyphenyl)-3-methyl-3,5-dihydro-4H -imidazol-4-one;
2-amino-5-(1-benzoylpiperidin-4-yl)-5-[3-(but-3-ynyloxy)phenyl]-3-methyl-3,5-dihydro -4H-imidazol-4-one;
2-amino-5-(1-benzoylpiperidin-4-yl)-5-[3-(cyclopropylmethoxy)phenyl]-3-methyl-3,5-dihydro-4H-imidazol-4-one;
N-{3-[2-amino-4-(1-benzoylpiperidin-4-yl)-1-methyl-5-oxo-4,5-dihydro-1H -imidazol-4-yl]phenyl}-2-methoxyacetamide;
3-[2-amino4-(1-benzoylpiperidin-4-yl)-1-methyl-5-oxo-4,5-dihydro-1H -imidazol-4-yl]-N-isobutylbenzamide;
ethyl 3-(2-amino-1-methyl-5-oxo4-phenyl4,5-dihydro-1H-imidazol-4-yl)piperidine-1-carboxylate;
2-amino-5-[1-(2-furoyl)piperidin-3-yl]-3-methyl-5-phenyl-3,5-dihydr-4H-imidazol -4-one;
2-amino-5-[1-(isoxazol-5-yl)piperidin-3-yl]-3-methyl-5-phenyl-3,5-dihydro-4H -imidazol-4-one;
2-amino-3-methyl-5-phenyl-5-[1-(trifluoroacetyl)piperidin-3-yl]-3,5-dihydro-4H -imidazol-4-one;
2-amino-5-[l-(cyclopentylcarbonyl)piperidin-3-yl]-3-methyl-5-phenyl-3,5-dihydro-4H -imidazol-4-one;
5-[1-(1-adamantylcarbonyl)piperidin-3-yl]-2-amino-3-methyl-5-phenyl-3,5-dihydr-4H -imidazol-4-one;
2-amino-5-(1-benzoylpiperidin-3-yl)-3-methyl-5-phenyl-3,5-dihydro-4H-imidazol-4-one;
2-amino-3-methyl-5-phenyl-5-[1-(thien-2-ylcarbonyl)piperidin-3-yl]-3,5-dihydro-4H -imidazol-4-one;
2-amino-5-[1-(3-methoxybenzoyl)piperidin-3-yl]-3-methyl-5-phenyl-3,5-dihydro-4H -imidazol-4-one;
2-amino-3-methyl-5-[1-(3-methylbutanoyl)piperidin-3-yl]-5-phenyl-3,5-dihydro-4H -imidazol-4-one;
4-[3-(2-amino-1-methyl-5-oxo-4-phenyl4,5-dihydro-1H-imidazol-4-yl)piperidin -1-yl]-4-oxobutanoic acid;
{2-[3-(2-amino-1-methyl-5-oxo-4-phenyl-4,5-dihydro-1H-imidazol-4-yl)piperidin -1-yl]-2-oxoethoxy}acetic acid;
5-[3-(2-amino-1-methyl-5-oxo-4-phenyl-4,5-dihydro-1H-imidazol-4-yl)piperidin -1-yl]-5-oxopentanoic acid;
2-amino-5-(1-benzoylpiperidin-4-yl)-5-[3-(2-fluoropyridin-3-yl)phenyl]-3-methyl-3,5-dihydro-4H-imidazol-4-one;
2-amino-5-(1-benzoylpiperidin-4-yl)-3-methy-5-(3-pyrimidin-5-ylphenyl)-3,5-dihydro -4H-imidazol-4-one;
2-amino-5-(1-benzoylpiperidin-4-yl)-5-[3-(5-methoxypyridin-3-yl)phenyl]-3-methyl-3,5-dihydro-4H-imidazol-4-one;
2-amino-5-(1-benzoylpiperidin-4-yl)-5-(3′-methoxybiphenyl-3-yl)-3-methyl-3,5-dihydro-4H-imidazol-4-one;
2-amino-5-(1-benzoylpiperidin-4-yl)-5-(3′-fluorobiphenyl-3-yl)-3-methyl-3,5dihydro -4H-imidazol-4-one;
2-amino-5-(1-benzoylpiperidin-4-yl)-5-(3′-chlorobiphenyl-3-yl)-3-methyl-3,5-dihydro -4H-imidazol-4-one;
2-amino-5-(1-benzoylpiperidin-4-y)-5-(2′,5′-difluorobiphenyl-3-yl)-3-methyl -3,5-dihydro-4H-imidazol-4-one;
2-amino-5-(1-benzoylpiperidin-4-yl)-5-(3′,5′-difluorobiphenyl-3-yl)-3-methyl -3,5-dihydro-4H-imidazol-4-one; or
   a tautomer thereof or a stereoisomer thereof or a pharmaceutically acceptable salt thereof.

Compounds of the invention may be conveniently prepared using conventional synthetic methods and, if required, standard separation and isolation techniques. For example, compounds of formula I wherein R is H (Ia) may be prepared by reducing a compound of formula 11 using standard reduction techniques such as catalytic hydrogenation. Compounds of formula I wherein R is other than H (Ib) may be prepared by coupling a compound of formula Ia with a reagent, such as an alkyl- aryl- or acylhalide (R-Hal) in the presence of a base. The reactions are shown in Flow Diagram I wherein Hal represents Cl, Br or I.

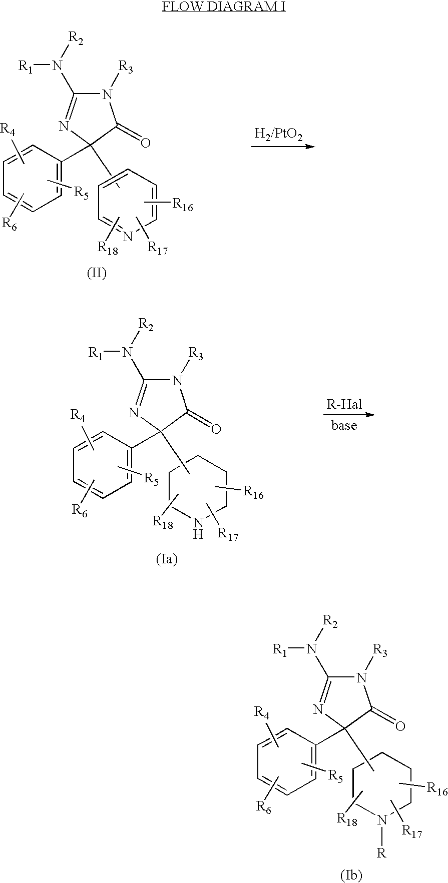

Reagents suitable for converting compounds of formula Ia to compounds of formula Ib include alkyl- or arylhalides, alkyl or aryl acid chlorides, anhydrides, carboxylic acids or the like. Compounds of formula II and their preparation are described in copending patent application Ser. No. 60/695305, which application is incorporated herein by reference thereto.

For example, compounds of formula II wherein R₁ and R₂ are H (Ia) may be prepared by reacting a bromobenzene compound of formula III with trifluoromethylsilylacetylene to give the arylalkyne of formula IV; reacting the formula IV alkyne with a bromopyridine compound of formula V to give the alkyne compound of formula VI; oxidizing the formula VI alkyne with an oxidizing agent such as Pd(II)CI/DMSO, N-bromosuccinimide/DMSO, ozone, sodium periodate with ruthenium (IV) oxide hydrate, sulfur trioxide, KMnO₄, I₂/DMSO, or combinations thereof, preferably KMnO₄, to give the diketone of formula VII; and reacting said formula VII diketone with an aminoguanidine derivative of formula VIII in the presence of a base, such as a metal carbonate, to give the desired formula IIa compound. The reaction is shown in flow diagram II.

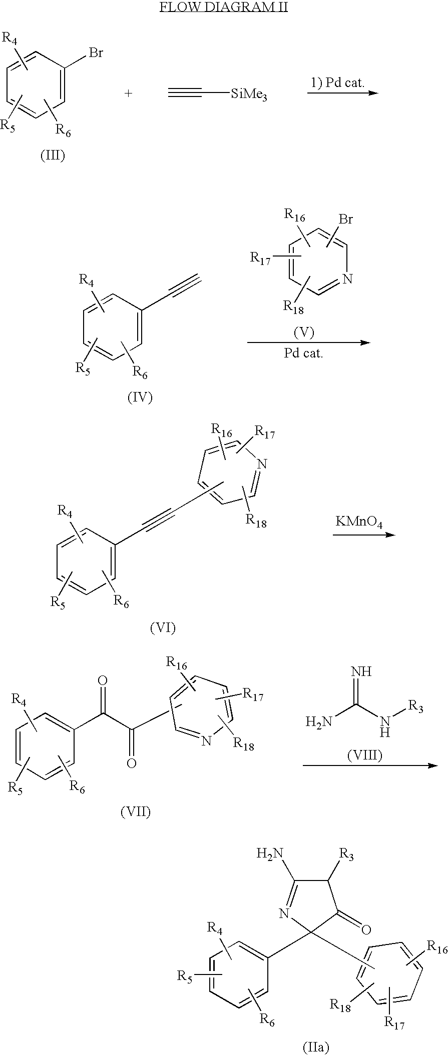

Advantageously, the compounds of formula I act as BACE inhibitors for the treatment or prevention of β-amyloid deposits and neurofibrillary tangles associated with such diseases as Alzheimer's disease, Trisomy 21 (Down's Syndrome), Hereditary Cerebral Hemorrhage with Amyloidosis of the Dutch-type (HCHWA-D), and other neurodegenerative disorders. Accordingly, the present invention provides methods for modulating BACE and treating, preventing, or ameliorating β-amyloid deposits and neurofibrillary tangles associated with diseases and disorders such as Alzheimer's disease, Trisomy 21 (Down's Syndrome), Hereditary Cerebral Hemorrhage with Amyloidosis of the Dutch-type (HCHWA-D), and other neurodegenerative disorders. Such methods generally involve administering to a patient suspected of suffering from or being susceptible to the disease or injury an effective amount of a compound of formula 1. Also according to the present invention there is provided a method of treating Alzheimer's disease and related senile dementia's in humans or other mammals which comprises administering to a human or other mammal an effective amount of a compound of the present invention.

The present invention also provides methods for modulating (and, preferably, inhibiting) the activity of BACE, comprising administering to a patient and/or contacting a receptor thereof with an effective amount of at least one compound of Formula I. Certain methods further comprise determining BACE activity, either before or after said contacting step.

The present invention also provides methods of ameliorating β-amyloid deposits in a mammal, comprising administering to said mammal an effective amount of at least one compound of Formula I. Further methods ameliorate neurofibrillary tangles in a mammal, and comprise administering to said mammal an effective amount of at least one compound of Formula I.

Also provided are methods of ameliorating symptoms of Alzheimer's disease, cognitive impairment, Down's Syndrome, HCHWA-D, cognitive decline, senile dementia, cerebral amyloid angiopathy, degenerative dementia, or other neurodegenerative disorders in a mammal, comprising administering to said mammal an effective amount of at least one compound of Formula I.

As used in accordance with this invention, the term “providing,” with respect to providing a compound or substance covered by this invention, means either directly administering such a compound or substance, or administering a prodrug, derivative, or analog which will form the effective amount of the compound or substance within the body. This invention also covers providing the compounds of this invention to treat the disease states disclosed herein that the compounds are useful for treating.

The terms “administer”, “administering”, or “administration”, as used herein, refer to either directly administering a compound or composition to a patient, or administering a prodrug derivative or analog of the compound to the patient, which will form an equivalent amount of the active compound or substance within the patient's body.

The term “patient”, as used herein, refers to a mammal, preferably a human.

The terms “effective amount”, “therapeutically effective amount” and “effective dosage” as used herein, refer to the amount of a compound that, when administered to a patient, is effective to at least partially ameliorate (and, in preferred embodiments, cure) a condition from which the patient is suspected to suffer. It is understood that the effective dosage of the active compounds of this invention may vary depending upon the particular compound utilized, the mode of administration, the condition, and severity thereof, of the condition being treated, as well as the various physical factors related to the individual being treated. For treating Alzheimer's disease and other related senile dementia's, generally, satisfactory results may be obtained when the compounds of this invention are administered to the individual in need at a daily dosage of from about 0.1 mg to about 1 mg per kilogram of body weight, preferably administered in divided doses two to six times per day, or in a sustained release form. For most large mammals, the total daily dosage is from about 3.5 mg to about 140 mg preferably from about 3.5 to about 5 mg. In the case of a 70 kg human adult, the total daily dose will generally be from about 7 mg to about 70 mg and may be adjusted to provide the optimal therapeutic result. This regimen may be adjusted to provide the optimal therapeutic response.

The present invention also provides a pharmaceutical composition which comprises an effective amount of a compound of formula I and a pharmaceutically acceptable carrier.

The term “carrier”, as used herein, shall encompass carriers, excipients, and diluents. Examples of carriers are well known to those skilled in the art and are prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances which may also act as flavoring agents, lubricants, solubilizers, suspending agents, fillers, glidants, compression aids, binders or tablet-disintegrating agents or encapsulating materials. They are formulated in conventional manner, for example, in a manner similar to that used for known antihypertensive agents, diuretics and β-blocking agents. Oral formulations containing the active compounds of this invention may comprise any conventionally used oral forms, including tablets, capsules, buccal forms, troches, lozenges and oral liquids, suspensions or solutions. In powders, the carrier is a finely divided solid, which is an admixture with the finely divided active ingredient. In tablets, the active ingredient is mixed with a carrier having the necessary compression properties in suitable proportions and compacted in the shape and size desired. The powders and tablets preferably contain up to 99% of the active ingredient.

Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colliodol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or fruit juice, containing appropriate solubilizers or emulisifiers as needed.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. Such administrations may be carried out using the present compounds, or pharmaceutically acceptable salts thereof, in lotions, creams, foams, patches, suspensions, solutions, and suppositories (rectal and vaginal).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. In therapeutic application, compounds of the present invention are provided to a patient already suffering from a disease in an amount sufficient to cure or at least partially ameliorate the symptoms of the disease and its complications. An amount adequate to accomplish this is defined as a “therapeutically effective amount”. The dosage to be used in the treatment of a specific case must be subjectively determined by the attending physician. The variables involved include the specific condition and the size, age and response pattern of the patient.

In some cases it may be desirable to administer the compounds directly to the airways in the form of an aerosol. For administration by intranasal or intrabrochial inhalation, the compounds of this invention may be formulated into an aqueous or partially aqueous solution.

The compounds of this invention may be administered parenterally or intraperitoneally. Solutions or suspensions of these active compounds as a free base or pharmaceutically acceptable salt may be prepared in water suitably mixed with a surfactant such as hydroxyl-propylcellulose. Dispersions may also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The compounds of this invention can be administered transdermally through the use of a transdermal patch. For the purposes of this disclosure, thransdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream, such as a semi-permeable membrane covering a reservoir containing the active ingredient with or without a carrier, or a matrix containing the active ingredient. Other occlusive devices are known in the literature.

In certain embodiments, the present invention is directed to prodrugs. Various forms of prodrugs are known in the art, for example, as discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al. (ed.), “Design and Application of Prodrugs”, Textbook of Drug Design and Development, Chapter 5, 113-191 (1991), Bundgaard, et al., Journal of Drug Deliver reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975).

It is understood that the dosage, regimen and mode of administration of these compounds will vary according to the malady and the individual being treated and will be subject to the judgment of the medical practitioner involved. It is preferred that the administration of one or more of the compounds herein begin at a low dose and be increased until the desired effects are achieved.

For a more clear understanding, and in order to illustrate the invention more clearly, specific examples thereof are set forth hereinbelow. The following examples are merely illustrative and are not to be understood as limiting the scope and underlying principles of the invention in any way. Various modifications of the invention, in addition to those described herein, will be apparent to those skilled in the art from the foregoing description. Such modifications are also intended to fall within the scope of the appended claims.

Unless otherwise stated, all parts are parts by weight. The following abbreviations are used: DIPEA is N,N-diisopropylethylamine; DMF is N,N-dimethyl formamide; DMSO is dimethylsulfoxide; EDCI is 1-(3-dimethylaminopropyl)-3-ethylcarbodiimide hydrochloride; EtOAc is ethyl acetate; TEA is triethylamine; THF is tetrahydrofuran; HNMR is proton nuclear magnetic resonance, and MS is mass spectroscopy with (+) referring to the positive mode which generally gives a M+1 (or M+H) absorption where M=the molecular mass.

### EXAMPLE 1

### Preparation of 2-Amino-5-(1,1′-biphenyl-3-yl)-3-methyl-5-piperidin-4-yl-3,5-dihydro-4H-imidazol-4-one dihydrochloride

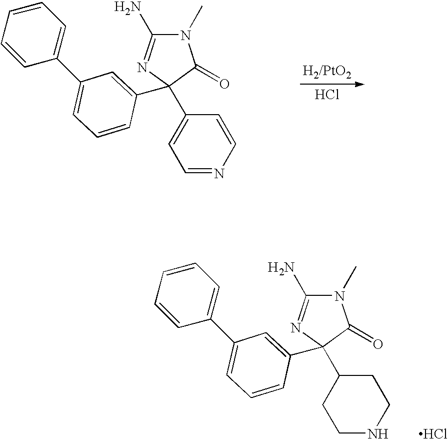

A suspension of 2-amino-5-(1,1′-biphenyl-3-yl)-3-methyl-5-pyridin-4-yl-3,5-dihydro-4H-imidazol-4-one (1.3 g, 3.8 mmol) in ethanol is treated with conc. HCl (0.47 mL, 5.7 mmol) followed by PtO₂ (84 mg). The reaction mixture is placed on a Parr shaker under hydrogen (50 psi) and hydrogenated for 18 h. Additional conc. HCl (0.16 mL, 1.9 mmol) is added and the hydrogenation is continued for 2 h. The precipitated solid is collected by filtration. This solid (with the catalyst) is dissolved in methanol and filtered to remove the catalyst. The filtrate is concentrated to dryness to give the title compound (0.95 g, 59%) as a solid, mp 223-226° C., MS(+) ES: 349 (M+H)⁺.

### EXAMPLE 2

### Preparation of 2-Amino-5-(1,1′-biphenyl-3-yl)-5-(1-isobutyrylpiperidin-4-yl)-3-methyl-3,5-dihydro-4H-imidazol-4-one

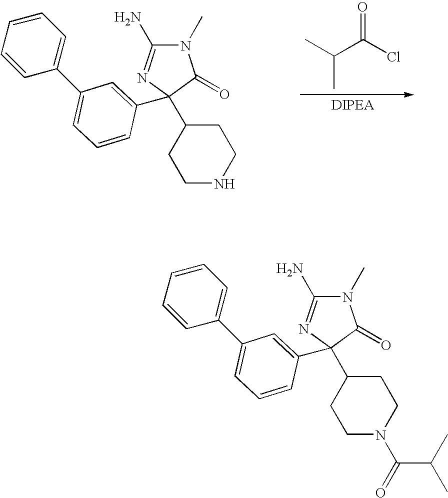

A solution of 2-amino-5-(1,1′-biphenyl-3-yl)-3-methyl-5-piperidin-4-yl-3,5-dihydro-4H-imidazol-4-one (69 mg, 0.2 mmol) in DMF is treated with 2-methyl -propanoyl chloride (21 mg, 0.2 mmol) and DIPEA (38 mg, 0.3 mmol) at room temperature. After stirring for 3 h, the reaction is quenched with water and extracted with ethyl acetate. The combined extracts are washed with water, brine, dried (MgSO₄) and concentrated. The resultant residue is purified by chromatography (silica gel, CH₂Cl₂/2M NH₃ in MeOH: 95/5) to afford the title compound (60 mg, 72%) as a white solid, mp 131-134° C., MS (+) ES: 419 (M+H)⁺.

### EXAMPLE 3

### Preparation of 2-Amino-5-(1,1′-biphenyl-3-yl)-3-methyl-5-[1-(thien-2-ylcarbonyl) -piperidin4-yl]-3,5-dihydro-4H-imidazol-4-one

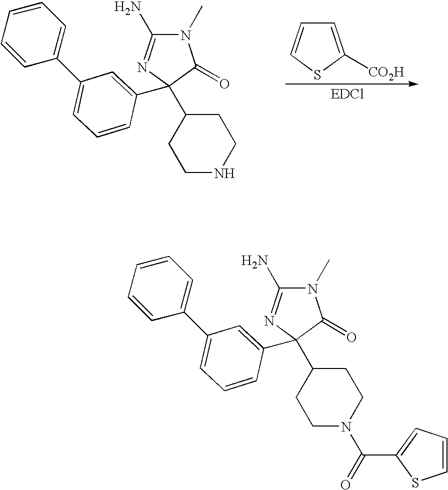

To a suspension of 2-amino-5-(1,1′-biphenyl-3-yl)-3-methyl-5-piperidin-4-yl -3,5-dihydro-4H-imidazol-4-one (obtained by dissolving the corresponding hydrochloride salt in methanol, neutralizing with 2M NH3/MeOH and evaporation of the mixture to dryness) (80 mg, 0.18 mmol, assuming 2 equiv of remaining NH₄Cl in the mixture) in CHCl₃ is added 2-thiophenecarboxylic acid (23 mg, 0.18 mmol) at room temperature. The mixture is stirred for 5 minutes and 1-(3-dimethylaminopropyl) -pyl)-3-ethylcarbodimide hydrochloride (51 mg, 0.26 mmol) is added. After stirring for 2 h, the reaction is quenched with saturated aqueous Na₂CO₃ and extracted with ethyl acetate. The combined extracts are washed sequentially with saturated aqueous Na₂CO₃ and brine, then dried (MgSO₄) and concentrated. The crude material is purified by chromatography (silica gel, CH₂Cl₂/2M NH₃ in MeOH: 92/8) to afford the title compound (51 mg, 63%) as a white solid, mp: 135-137° C., MS (+) ES: 459 (M+H)⁺.

### EXAMPLES 4-22

### Preparation of 2-Amino-5-(1,1′-biphenyl-3-yl)-5-(1-substituted-piperidin-4-yl)-3-methyl-3,5-hydro-4H-imidazol-4-one

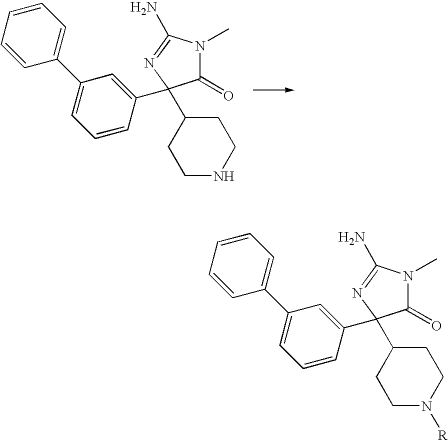

Using essentially the same procedures described in Examples 2 and 3 and employing the appropriate reagent, i.e. acid, acid chloride, sulfonyl chloride or alkyl chloride compounds shown in Table I are obtained and identified by NMR and mass spectral analyses.

TABLE I

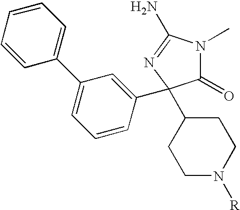

| Ex. | | mp | |
|---|---|---|---|
| No. | R | ° C. | M + H |
| 4 | benzoyl | 136-137 | 453 |
| 5 | 3-methoxybenzoyl | 131-134 | 483 |
| 6 | (benzyloxy)acetyl | 121-124 | 497 |
| 7 | 2-furoyl | 148-153 | 443 |
| 8 | phenylsulfonyl | 138-139 | 489 |
| 9 | 1-prop-2-ynyl | 118-119 | 387 |
| 10 | 3-furoyl | 93-95 | 443 |
| 11 | 2-chloro-6-methylisonicotinoyl | 145-147 | 502 |
| 12 | thien-3-ylcarbonyl | 138-140 | 459 |
| 13 | 3,4-dimethoxybenzoyl | 139-141 | 513 |
| 14 | 1,3-benzodioxol-5-ylcarbonyl | 133-135 | 497 |
| 15 | 1-naphthoyl | 161-163 | 303 |
| 16 | 4-cyanobenzoyl | 152-154 | 478 |
| 17 | 3-cyanobenzoyl | 141-143 | 478 |
| 18 | 2-cyanobenzoyl | 144-146 | 478 |
| 19 | 2-methoxybenzoyl | 141-143 | 482 |
| 20 | 4-methoxybenzoyl | 138-140 | 482 |
| 21 | 4-propylbenzoyl | 133-135 | 495 |
| 22 | 4-propoxybenzoyl | 131-133 | 511 |

### EXAMPLE 23

### Preparation of 2-Amino-5-(3-cyclohexylphenyl)-3-methyl-5-piperidin-4-yl-3,5-dihydo-4H-imidazol4-one hydrochloride

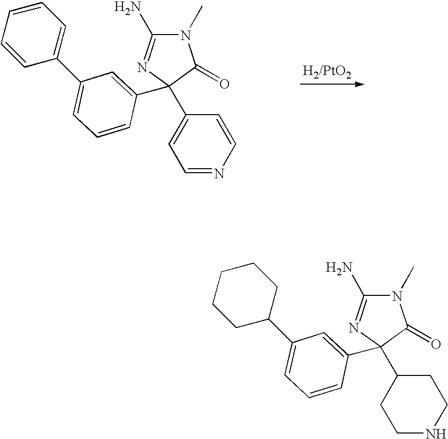

To a suspension of 2-amino-5-(1,1′-biphenyl-3-yl)-3-methyl-5-pyridin-4-yl-3,5-dihydro-4H-imidazol-4-one (0.71 g, 2.08 mmol) in ethanol is added conc. HCl (0.26 mL, 3.12 mmol) followed by PtO₂ (91 mg). The reaction mixture is placed on a Parr shaker under hydrogen (50 psi) and hydrogenated for 48 h. The catalyst is removed by filtration and the filtrate is concentrated to dryness to give the title compound (0.87 g, 96%) as a solid, mp 212-215° C., MS(+) ES: 355 (M+H)⁺.

### EXAMPLES 24 and 25

### Preparation of 2-Amino-5-(1-substitutedpiperidin-4-yl)-5-(3-cyclohexylphenyl) -3-methyl-3.5-dihydro-4H-imidazol-4-one

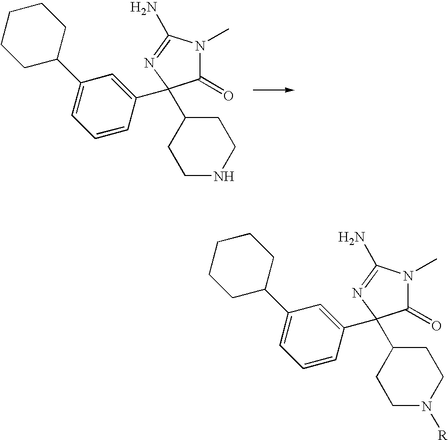

Using essentially the same procedures described in Example 2 and employing the appropriate acid chloride, the compounds shown in Table II are obtained and identified by NMR and mass spectral analyses.

TABLE II

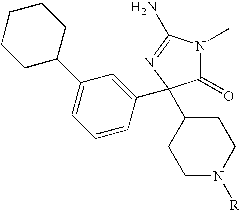

| Ex. | | mp | |
|---|---|---|---|
| No. | R | ° C. | M + H |
| 24 | benzoyl | 189-190 | 459 |
| 25 | acetyl | gum | 397 |

### EXAMPLE 26

### Preparation of 2-Amino-3-methyl-5-phenyl-5-piperidin-3-yl-3,5-dihydro-4H -imidazol-4-one

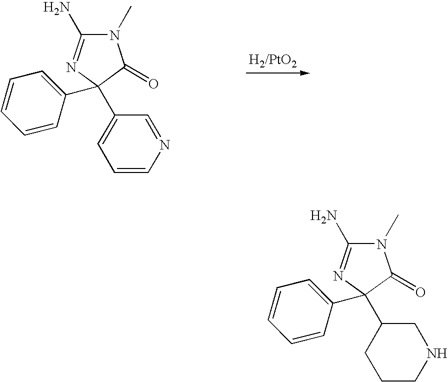

A mixture of 2-amino-3-methyl-5-phenyl-5-pyridin-3-yl-3,5-dihydro-4H -imidazol-4-one (4.9 g, 18.05 mmol), PtO₂ (0.24 g) and 4 M HCl (9 ml) ethanol is placed on a Parr shaker under hydrogen (48 psi) and hydrogenated overnight. After filtrating off the catalyst, the filtrate is neutralized with saturated aqueous Na₂CO₃to pH˜10 and concentrated to dryness to give the title compound as a white solid (contained a mixture of Na₂CO₃ and NaCl salts) (6.7 g). MS(+) ES: 273 (M+H)⁺.

### EXAMPLE 27

### Preparation of 2-Amino-3-methyl-5-phenyl-5-piperidin-4-yl-3,5-dihydro-4H -imidazol-4-one

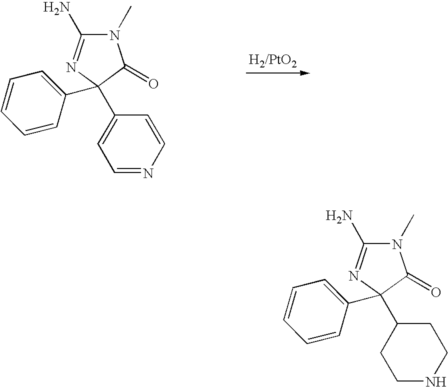

A mixture of 2-amino-3-methyl-5-phenyl-5-pyridin4-yl-3,5-dihydro-4H -imidazol-4-one (533 mg, 2.00 mmol), PtO₂ (0.57 mg) and acetic acid (3 ml) in ethanol is placed on a Parr shaker under hydrogen (50 psi) and hydrogenated for 24 h. The reaction mixture is treated with conc. HCl (pH=˜3) and PtO₂ (227 mg). The hydrogenation is continued for 48 h. The catalyst is removed by filtration and the filtrate is neutralized with conc. NH₄OH. The EtOH is removed and the residue is extracted with 4/1 CH₂Cl₂/PrOH. The combined extracts are washed with H₂O, brine, dried (MgSO₄), filtered and concentrated to dryness to give the title compound (122 mg, 22%) as a white solid, mp 269-271° C., MS(−) ES: 271 (M+H)⁻.

### EXAMPLE 28

### Preparation of N-(3-Ethynylphenyl)-2-methoxyacetamide

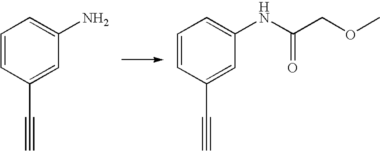

A cooled solution of 3-ethynylphenylamine (7.02 g, 60 mmol) and TEA (7.28 g, 72 mmol) in methylene chloride is treated dropwise with a solution of methoxyacetyl chloride (7.8 g, 72 mmol) in methylene chloride over a period of 30 min at 0° C., allowed to warm to room temperature, stirred overnight and concentrated in vacuo. The resultant residue is partitioned between water and ethyl acetate. The organic phase is separated, washed sequentially with saturated NaHCO₃ and H₂O, dried over MgSO₄ and evaporated to dryness to afford the title compound as a colorless oil, 10.2 g (90% yield), ¹HNMR (CDCl3): δ (ppm) 3.04 (s, 1H,), 3.48 (s, 3H), 3.98 (s, 2H), 7.24 (m, 2H), 7.61 (d, 1H), 7.66 (s, 1H), 8.21 (s, b, 1H).

### EXAMPLE 29

### Preparation of 2-Methoxy-N-(3-pyridin-4-ylethynylphenyl)acetamide

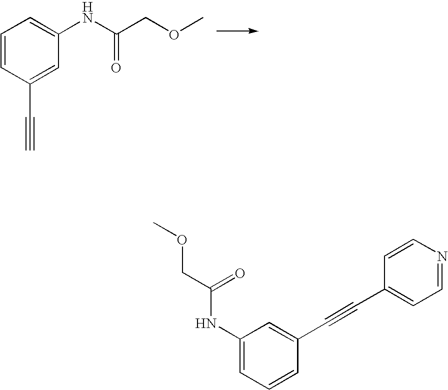

A mixture of 4-bromopyridine hydrochloride (10.40 g, 54 mmol), Cul (201 mg), Pd(PPh₃)₂Cl₂ (1.13 g, 1.62 mmol) and triethyl amine (38 mL) is stirred for 30 minutes at room temperature, treated with a solution of N-(3-ethynylphenyl)-2-methoxyacetamide (10.2 g, 54 mmol) in DMF, heated at 65-70° C. for 12 h, cooled to room temperature and partitioned between water and EtOAc. The organic layer is separated, dried over MgSO₄ and concentrated in vacuo. The resultant residue is purified by flash chromatography (silica gel, EtOAc: 100%) to afford the title compound as a solid, 8.0 g (57% yield), ¹HNMR (CDCl3): δ (ppm) 3.50 (s, 3H), 4.02 (s, 2H), 7.31-7.33 (m, 4H), 7.45 (b, 2H), 7.56 (d, 1H), 7.85 (s, 1H), 8.30 (s, 1H).

### EXAMPLE 30

### Preparation of 2-Methoxy-N-[-3-(2-oxo-2-pyridin-4-yl-acetyl)-phenyl]acetamide

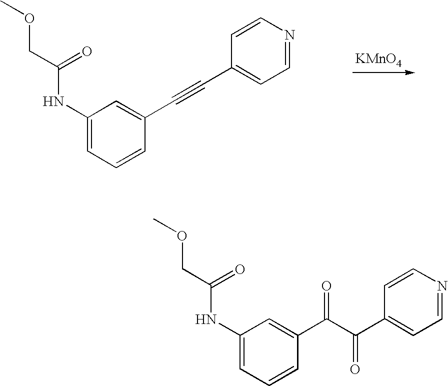

A solution of 2-methoxy-N-(3-pyridin-4-ylethynyl-phenyl)-acetamide (8.0 g, 30 mmol) in acetone is treated, with stirring, with a solution of MgSO₄ (5.51 g, 46 mmol) and NaHCO₃ (1.51 g, 18 mmol) in water, followed by treatment, in one portion, with KMnO₄ (10.43 g, 66 mmol). After stirring for 5 minute, the reaction mixture is extracted with ether. The combined extracts are dried over MgSO₄ and concentrated to dryness to afford the title compound as a solid, 2.7 g (30% yield), ¹HNMR (CDCl3): δ (ppm) 3.50 (s, 3H), 4.02 (s, 2H), 7.51 (t, 1H) 7.71 (d, 2H), 7.76 (d, 2H) 8.06 (d, 1H), 8.08 (s, 1H), 8.43 (s,1H), 8.86 (d, 2H).

### EXAMPLE 31

### Preparation of N-[3-(2-Amino-1-methyl-5-oxo-4-pyridin-4-yl4,5-dihydro-1H -imidazol-4-yl)phenyl]-2-methoxyacetamide

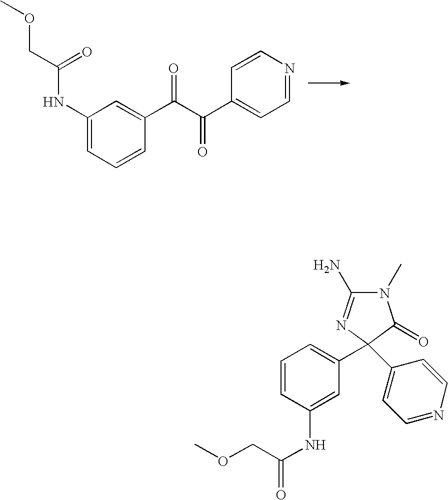

A mixture of 2-methoxy-N-[-3-(2-oxo-2-pyridin-4-yl-acetyl)-phenyl]acetamide (2.7 g, 9 mmol), methylguanidine (1.98 g, 18 mmol) and Na₂CO₃ (2.86 g, 27.2 mmol) in ethanol and water is heated at reflux temperature for 3 h and concentrated in vacuo. The resultant residue is purified by flash chromatography (silica gel, EtOAc/2.0M ethanolic NH₃: 90/10 to 80/20) to afford the title compound as a solid, 1.5 g (47% yield), mp 92-93° C.; MS (+) ES: 394 [M+H]⁺.

### EXAMPLE 32

### Preparation of N-[3-(2-Amino-1-methyl-5-oxo4-piperldin-4-yl-4,5-dihydro-1H -imidazol-4-yl)phenYl]-2-methoxyacetamide

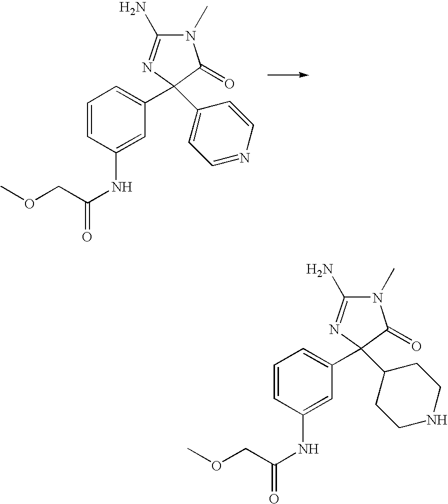

A mixture of N-[3-(2-amino-1-methyl-5-oxo-4-pyridin-4-yl-4,5-dihydro-1H -imidazol-4-yl)phenyl]-2-methoxyacetamide (353 mg, 1.0 mmol), PtO₂ (40 mg) and concentrated hydrochloride acid (0.17 mL, 2.0 mmol) is hydrogenated at 45 psi for 24 h at ambient temperature. The reaction mixture is filtered and the filtrate is concentrated to dryness. The resultant residue is dissolved in ethanol and stirred with Amberlyst A-26(OH) ion exchange resin (1.0 g) for 24 h and filtered. The filtrate is concentrated to dryness to afford the title compound as a solid, 340 mg (95% yield), mp 170-174° C., MS (+) ES: 360 [M+H]⁺.

### EXAMPLE 33

### Preparation of N-[3-(2-Amino-{1-]4-(benzyloxy)benzoyl]piperidin-4-yl}-1-methyl-5-oxo-4,5-dihydro-1H-imidazol-4-yl)phenyl]-2-methoxyacetamide

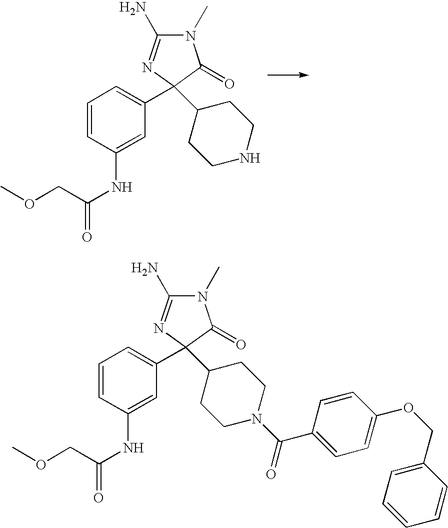

A cooled solution of N-[3-(2-amino-1-methyl-5-oxo-4-piperidin-4-yl-4,5-dihydro-1H imidazol-4-yl)phenyl]-2-methoxyacetamide (180 mg, 0.5 mmol) and p-benzyloxybenzoic acid (114 mg, 0.5 mmol) in methylene chloride and DMF is treated portionwise with 1-(3-dimethylaminopropyl)-3-ethylcarbodiimide hydrochloride (105 mg, 0.55 mmol) at 0° C., stirred for 2 h at 0° C.and for 12 h at room temperature and concentrated in vacuo. The resultant residue is purified by flash chromatography (silica gel, EtOAc/2.0M ethanolic NH₃: 80/20) to afford the title compound as a white solid, (80% yield), mp 149-152° C., MS (+) ES: 570 [M+H]⁺.

### EXAMPLE 34

### Preparation of N-(3-{2-Amino-4-[1-(4-hydroxybenzoyl)piperidin4-yl]-1-methyl-5-oxo-4,5-dihydro-1H-imidazol-4-yl}phenyl)-2-methoxyacetamide

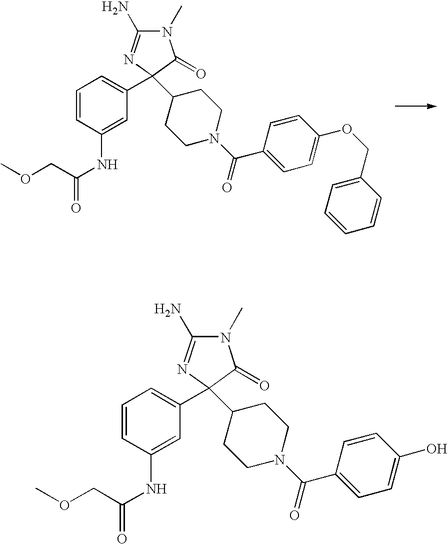

A mixture of N-(3-{2-amino-4-[1-(benzyloxybenzoyl)-piperidin-4-yl]-1-methyl -5-oxo-4,5-dihydro-1H imidazol-4-yl}phenyl)-2-methoxyacetamide (50 mg, 0.088 mmol) and Pd/C (5 mg) in ethanol is hydrogenated at 45 psi for 2 h and filtered. The filtrate is concentrated to dryness to afford the title compound as a solid, 40 mg (95% yield), mp 184-187° C., MS (+) ES: 480 [M+H]⁺.

### EXAMPLE 35

### Preparation of Methyl 4-{[4-(2-amino-4-{3-[(methoxyacetyl)amino]phenyl}-1-methyl-5-oxo-4,5-dihydro-1H-imidazol-4-yl)piperidin-1-yl]carbonyl}benzoate

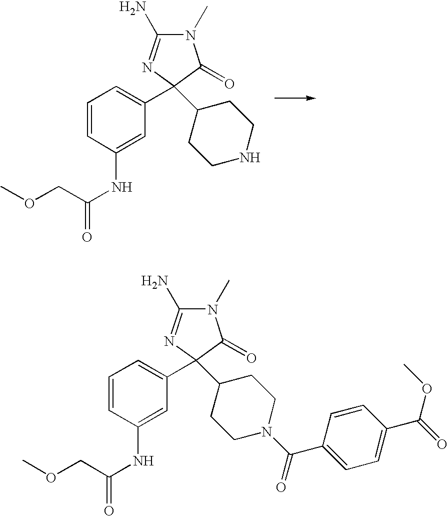

Using the essentially same procedure described in Example 33 and employing terephthalic acid monomethyl ester, the title compound is obtained as a solid, mp 161-163° C., MS (+) ES: 521 [M+H]⁺.

### EXAMPLE 36

### Preparation of Sodium 4-{[4-(2-Amino4-{3-[(methoxyacetyl)amino]phenyl}-1-methyl-5-oxo-4,5-dihydro-1H-imidazol-4-yl)piperidin-1-yl]carbonyl}benzoate

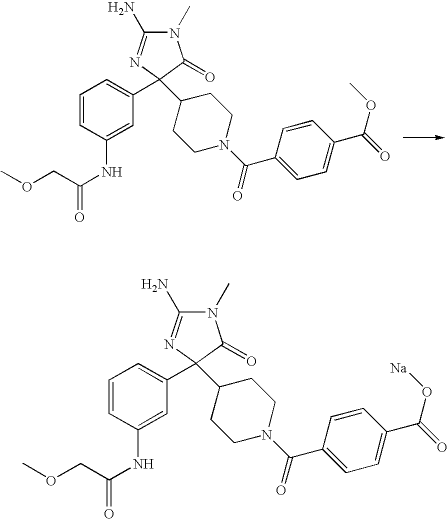

A solution of NaOH (7.06 mg, 0.177 mmol) in ethanol is treated with 4-(4-{2-amino4-[3-(2-methoxyacetylamino)phenyl]-1-methyl-5-oxo-4,5-dihydro-1H-imidazol -4-yl}piperidine-1-carbonyl)benzoic acid methyl ester (92 mg, 0.177 mmol), stirred for 48 h at room temperature and concentrated in vacuo. The resultant residue is dissolved in a small amount of CH₂Cl₂, treated with ether and filtered. The filtercake is dried to afford the title compound as a solid, 70 mg (75% yield), mp >250° C., MS (+) ES: 507 [M+H]⁺.

### EXAMPLES 37-58

### Preparation of N-(3-{2-Amino-4-[1-acylpiPeridin-4-yl]-1-methyl-5-oxo-4,5-dihydro-1H-imidazol-4-yl}phenyl)-2-methoxyacetamide

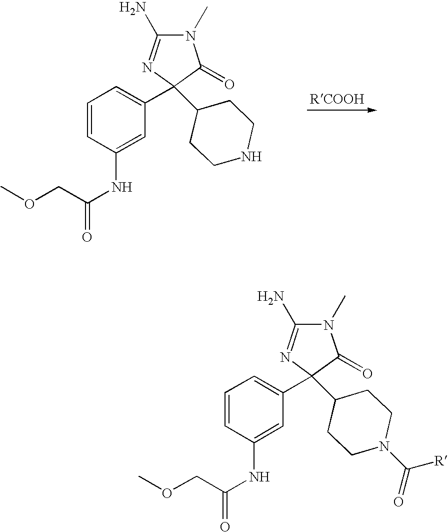

Using essentially the same procedure described in Example 33 and employing the appropriate acid, the compounds shown in Table III are obtained and identified by NMR and mass spectral analyses.

TABLE III

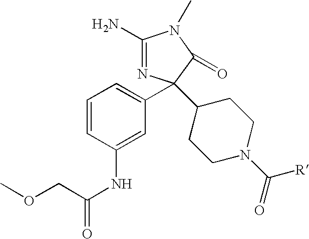

| Ex. | | mp | |
|---|---|---|---|
| No. | R′ | ° C. | M + H |
| 37 | cyclopropyl | >130 dec | 428 |
| 38 | cyclohexanemethyl | 131-133 | 484 |
| 39 | cyclohexyl | 145-147 | 469 |
| 40 | cyclopentyl | 134-136 | 456 |
| 41 | 2-cyclohexylethyl | 128-130 | 497 |
| 42 | isopropyl | 145-148 | 430 |
| 43 | 1-ethylpropyl | 102-105 | 458 |
| 44 | 5-oxotetrahydrofuran-2-yl | 118-120 | 472 |
| 45 | 2-chlorophenyl | 130-133 | 498 |
| 46 | 1-benzofuran-2-yl | 139-142 | 504 |
| 47 | 3-butyn-1-yl | 135-138 | 440 |
| 48 | 1-propylbutyl | gel | 486 |
| 49 | 3-methylbutyl | 105-107 | 458 |
| 50 | 3-fluorophenyl | 150-153 | 482 |
| 51 | 1,3-benzodioxol-5-yl | >160 dec. | 508 |
| 52 | 4-cyanophenyl | 160-164 | 489 |
| 53 | 3-furyl | 140-142 | 454 |
| 54 | 2-naphthyl | 168-170 | 514 |
| 55 | 2-thienyl | 148-152 | 470 |
| 56 | methoxymethyl | 79-80 | 432 |
| 57 | 5-bromo-3-pyridinyl | 147-150 | 544 |
| 58 | trifluoromethyl | 120-122 | 456 |

### EXAMPLE 59

### Preparation of N{3-[2-Amino-4-(1-benzylpiperidin-4-yl)-1-methyl-5-oxo-4,5-dihydro-1H-imidazol-4-yl]phenyl}-2-methoxyacetamide

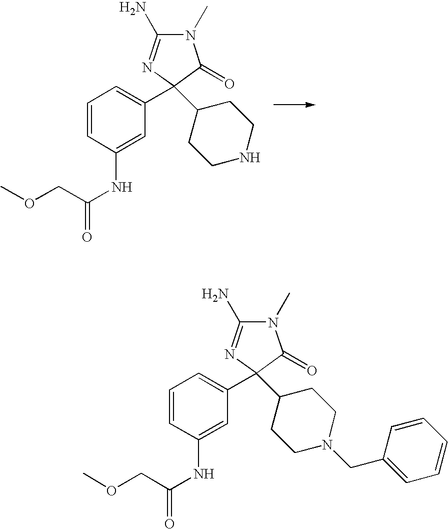

A mixture of N-[3-(2-amino-1-methyl-5-oxo4-pyperidin-4-yl-4,5-dihydro-1H imidazol-4-yl)phenyl]-2-methoxyacetamide (180 mg, 0.5 mmol), benzylbromide (85 mg, 0.5 mmol) and K₂CO₃ (138 mg, 1.0 mmol) in acetonitrile and ethanol is stirred at room temperature for 24 h and concentrated in vacuo. The resultant residue is purified by flash chromatography (silica gel, EtOAc/2.0M ethanolic NH₃: 80/20) to afford the title compound as a white solid, 102 mg (46% yield), mp 120-123° C., MS (+) ES: 450 [M+H]⁺.

### EXAMPLE 60

### Preparation of Ethyl 3-(2-Amino-1-methyl-5-oxo-4-phenyl-4,5-dihydro-1H -imidazol-4-yl)piperidine-1-carboxylate

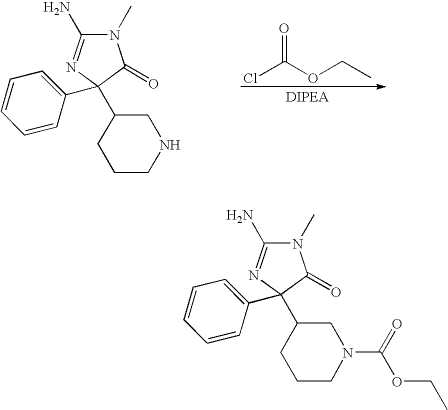

A solution of 2-amino-3-methyl-5-phenyl-5-(piperidin-3-yl)-3,5-dihydroimidazol-4-one (0.096 g, 0.35 mmol) in DMSO is treated sequentially with a solution of diisopropylethylamine (DIPEA) (0.5 mL) in THF and ethyl chloroformate (0.32 mmol), shaken for 16 h and concentrated in vacuo under a nitrogen stream.

The resultant residue is dissolved in DMSO and purified by preparative reverse phase HPLC¹ and characterized by LCMS² analysis, M+H 345, retention time 2.07 mmmin.
¹Gilson preparataive reverse phase HPLC system: YMC Pro C18, 20 mm×50 mm ID, 5 μM column; 2 mL injection; Solvent A: 0.02% NH₄OH/water; Solvent B: 0.02% NH₄OH/acetonitrile; Gradient: Time 0: 95% A; 2 min: 95% A; 14 min: 10% A; 16 min: 95% A; Flow rate 22.5 mUmin; Detection: 254 nm DAD

²LCMS Conditions: Hewlett Packard 1100 HPLC system; Waters Xterra MS C18, 2 mm (i.d.)×50 mm (length), 3.5 μm column, set at 50° C.; Flow rate 1.0 mL/min; Solvent A: 0.02% NH₄OH/water; Solvent B: 0.02% NH₄OH/acetonitrile; Gradient: Time 0: 10% B; 2.5 min: 90% B; 3 min: 90% B; Sample concentration: −2.0 mM; Injection volume: 5 μL; Detection: 220 nm, 254 nm DAD.

### EXAMPLES 61-73

### Preparation of 2-Amino-5-(phenyl)-5-(1-substituted-piperidin-3-yl)-3-methyl-3,5-dihydro-4H-imidazol-4-one

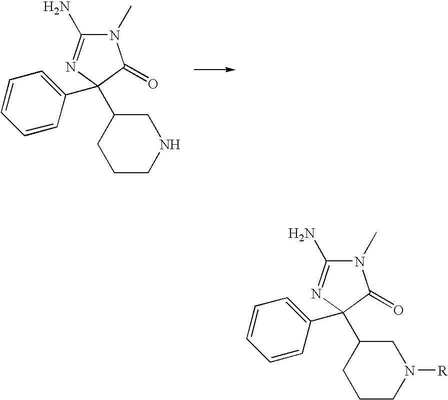

Using essentially the same procedure described in Example 60 and employing the appropriate reagent, i.e. acid, anhydride or acid chloride, the compounds shown in Table IV are obtained, and identified by LCMS analysis. HPLC and LCMS conditions are the same as those used in Example 60. The column heading RT designates retention time.

TABLE IV

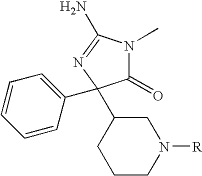

| Ex. | | | RT |
|---|---|---|---|
| No. | R | [M + H] | (min) |
| 61 | 2-furoyl | 367 | 1.85 |
| 62 | isoxazol-5-ylcarbonyl | 368 | 1.7* |
| 63 | trifluoroacetyl | 369 | 1.94 |
| 64 | cyclopentylcarbonyl | 369 | 2.1 |
| 65 | 1-adamantylcarbonyl | 435 | 2.6 |
| 66 | benzoyl | 377 | 1.93 |
| 67 | thien-2-ylcarbonyl | 383 | 1.92 |
| 68 | 3-methoxybenzoyl | 407 | 1.98 |
| 69 | 3-methylbutanoyl | 357 | 2.03 |
| 70 | 4-cyanobenzoyl | 402 | 1.84 |
| 71 | CO(CH₂)₂CO₂H | 373 | 1.64 |
| 72 | COCH₂OCH₂CO₂H | 89 | 1.59** |
| 73 | CO(CH₂)₃CO₂H | 387 | 1.68 |

*diastereomer, 2nd diastereomer rt is 1.31 min
**diastereomer, 2nd diastereomer rt is 1.68 min

### EXAMPLE 74

### Preparation of (1-Benzoylpiperidin-4-yl)methanol

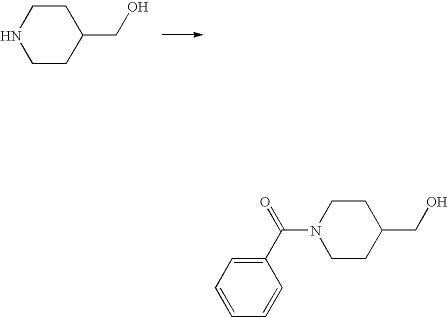

A cooled solution of piperidin-4-ylmethanol (2.6 g, 22.6 mmol) and triethyl amine (4.6 g, 45.2 mmol) in methelene chloride was treated dropwise with stirring with a solution of benzoyl chloride (3.16 g, 22.6 mmol) in methelene chloride over a period of 30 min. After addition is complete, the reaction mixture is allowed to warm to room temperature, stirred for 4 h at room temperature and concentrated under vacuum. The resultant residue is purified by flash chromatography (silica gel, EtOAc: 100%) to afford the title compound as an oil, 3.0 g (60% yield), which solidified upon standing. MS (+) ES: 220.1 (M+H)⁺, ¹HNMR (CDCl3) δ (ppm) 1.23 (b, 2H), 1.76 (b, 3H), 2.88 (d, 2H), 3.51 (d, 2H), 3.81 (b, 1H), 4.72 (s, 1H), 7.39 (m, 5H).

### EXAMPLE 75

### Preparation of 1-Benzoylpiperidine-4-carbaldehyde

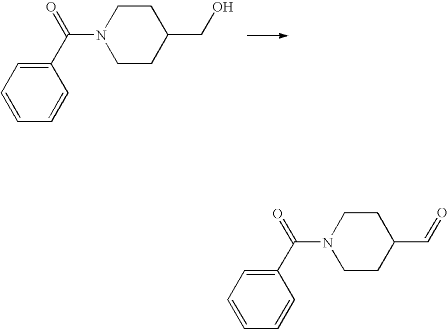

To a suspension of pyridinium chlorochromate (4.4 g, 20.5 mmol) in methelene chloride is added in one portion a solution of (1-benzoylpiperidin-4-yl)methanol (3.0 g, 13.7 mmol) in methylene chloride. The reaction mixture is stirred under nitrogen at room temperature for 90 min, diluted with ether and filtered through a pack of silica gel. The filtercake is washed with ethyl acetate. The filtrates are combined and concentrated to dryness to afford the title compound as an oil, 1.5 g (50% yield). MS (+) ES: 218 (M+H)⁺, ¹HNMR (DMSO-d₆) δ (ppm) 1.42 (b, 2H), 1.84 (b, 2H), 2.58 (m, 1H), 3.03 (b, 2H), 3.46 (b, 1H), 4.19 (s, 1H), 7.31-7.43 (m, 5H), 9.56 (s, 1H).

### EXAMPLE 76

### Preparation of 1-Benzoyl-4-ethynylpiperidine

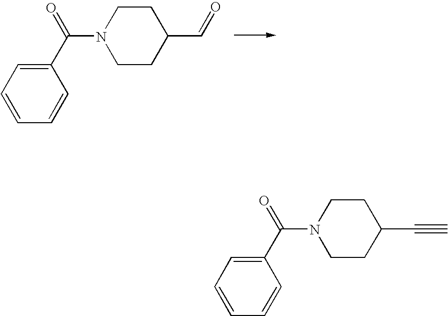

A stirred mixture of 1-benzoylpiperidine-4-carbaldehyde (1.3 g, 6 mmol) and K₂CO₃ (1.65 g, 12 mmol) in methanol is treated dropwise with dimethyl (1-diazo-2-oxopropyl)phosphonate (1.38 g, 7.2 mmol) over a 10 min. period, stirred for 4 h, diluted with ether and washed sequentially with 5% sodium bicarbonate and water. The organic phase is dried over MgSO₄ and concentrated in vacuo. The resultant residue is purified by flash chromatography using ethyl acetate/hexane (30/70) as eluent to afford the title compound as a white solid, 1.2 g (94% yield), mp 101-103° C. MS (+) ES: 214.1 (M+H)⁺, 1HNMR (DMSO-d6): 6(ppm) 1.45 (b, 2H), 1.74 (b, 2H), 2.62 (m, 1H), 3.17 (b, 2H), 3.40 (b, 1H), 3.91 (b, 1H), 7.30-7.42 (m, 5H).

### EXAMPLE 77

### Preparation of 1-Benzoyl-4-[(3-bromophenyl)ethynyl]piperidine

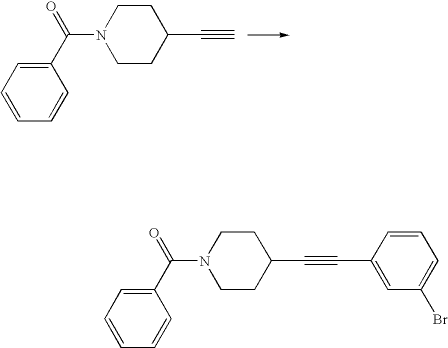

A mixture of 1-benzoyl4-ethynylpiperidine (852 mg, 4 mmol) and 1-bromo-3-iodobenzene (1.13 g, 4 mmol), Cul (38 mg, 0.2 mmol), Pd(PPh₃)₂Cl₂(184 mg, 0.16 mmol) in a mixture of triethyl amine (12 mL) and acetonitrile (6 mL) is heated at reflux temperature for 3 h, cooled to room temperature and evaporated under reduced pressure. The resultant residue is partitioned between water and EtOAc. The organic phase is separated, dried over MgSO₄ and concentrated in vacuo. This residue is purified by flash chromatography (silica gel, EtOAc/hexane: 20/80 to 50/50) to afford the title compound as a colorless oil, 1.1 g (75% yield). MS (+) ES: 368.0(M+H)⁺, 1HNMR (DMSO-d6): δ (ppm) 1.57 (b, 2H), 1.83 (b, 2H), 2.95 (m, 1H), 3.21 (b, 1H), 3.38 (b, 1H), 3.44 (b, 1H), 3.96 (b, 1H), 7.27 (t, 1H), 7.34-7.42 (m, 6H), 7.50 (d,1H), 7.57(s, 1H).

### EXAMPLE 78

### Preparation of 1-(1-Benzoylpiperidin-4-yl)-2-(3-bromophenyl)ethane-1,2-dione

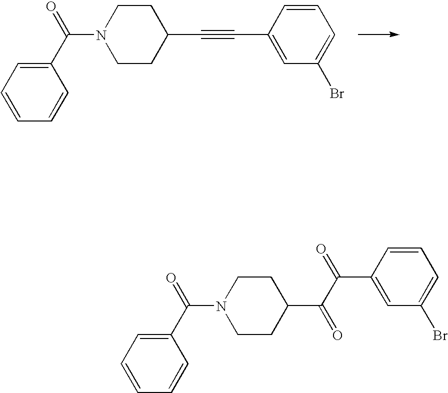

A solution of 1-benzoyl-4-[(3-bromophenyl)ethynyl]piperidine (1.1 g, 3 mmol) in acetone is treated with stirring with a solution of MgSO₄ (540 mg, 4.5 mmol) and NaHCO₃ (150 mg, 1.8 mmol) in water, treated in one portion with solid KMnO₄ (1.42 g, 9 mmol), stirred for 10 min and extracted with ether. The extracts are combined, dried over MgSO₄ and concentrated in vacuo to afford the title compound as a yellow oil 900 mg (76% yield). MS (+) ES: 400(M+H)⁺, 1HNMR (DMSO-d6): δ (ppm) 1.55 (b, 2H), 1.81 (b, 2H), 2.95 (b, 1H), 3.09 (b, 1H), 3.40 (m, 1H), 3.65 (b,1H), 4.42(b, 1H), 7.30-7.45 (m, 5H), 7.51 (t, 1H), 7.90 (m, 1H), 8.00 (s, 1H).

### EXAMPLE 79

### Preparation of 2-Amino-5-(1-benzoylpiperidin-4-yl)-5-(3-bromophenyl)-3-methyl -3,5-dihydro-4H-imidazol-4-one

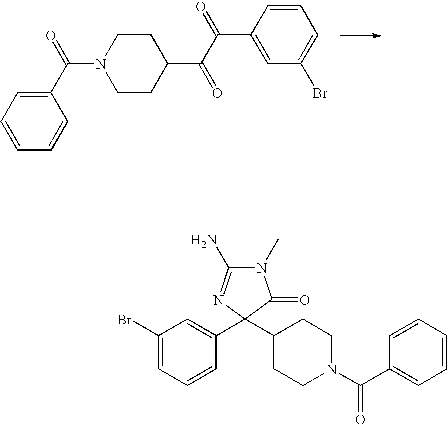

A mixture of 1-(1-benzoylpiperidin-4-yl)-2-(3-bromophenyl)ethane-1,2-dione (900 mg, 2.25 mmol), methylguanidine (493 mg, 4.5 mmol) and Na₂CO₃ (567 mg, 5.4 mmol) in ethanol is heated at reflux temperature for 3 h, cooled to room temperature and concentrated in vacuo. The resultant residue is purified by flash chromatography (silica gel, EtOAc/2.0M ethanolic NH₃: 95/5) to afford the title compound as a white solid, 700 mg (68% yield), mp >250° C. MS (+) ES: 455.1(M+H)⁺, 1HNMR (DMSO-d6): δ (ppm) 1.02-1.04 (b, 4H), 2.20 (b, 1H), 2.48-2.58 (b, 2H), 2.88 (s, 3H), 3.00(b, 1H), 3.55 (b, 1H), 4.33 (b, 1H), 6.70 (s, 2H), 7.27 (m, 2H), 7.41 (m, 3H), 7.61 (d,1H), 7.74 (s, 1H).

### EXAMPLES 80-87

### Preparation of 2-amino-5-(1-benzoylpiperidin-4-yl)-5-(3-substituted-phenyl)-3-methyl-3,5-dihydro-4H-imidazol-4-one Compounds

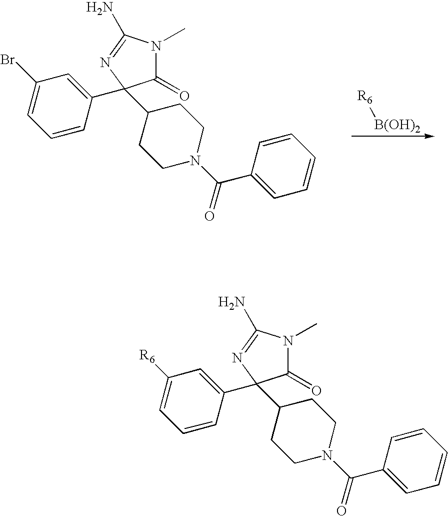

A mixture of an appropriately substituted boronic acid (R₄-B(OH)₂) (0.528 mmol), dichloro(triphenylphosphine)palladium (18.5 mg, 0.0264 mmol), triphenylphosphine (3.5 mg (0.0132 mmol) and sodium carbonate (83 mg, 0.8 mmol) is treated with a solution of 2-amino-5-(1-benzoylpiperidin-4-yl)-5-(3-bromophenyl)-3-methyl-3,5-dihydro-4H-imidazol-4-one in toluene/ethanol (1/1), heated to reflux temperature for 3 h, cooled to room temperature and concentrated in vacuo. The resultant residue is purified by chromatography (silica gel, EtOAc/2M ethanolic NH₃: 90/10) to afford the compounds shown in Table V. The compounds shown in Table V were identified by NMR and mass spectral analyses.

TABLE V

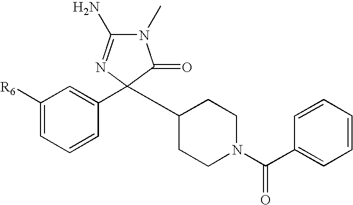

| Ex. | | mp | |
|---|---|---|---|
| No. | R6 | ° C. | [M + H] |
| 80 | 2-fluoropyridin-3-yl | 152-154 | 472.2 |
| 81 | 3-pyrimidin-5-yl | 156-158 | 455.2 |
| 82 | 5-methoxypyridin-3-yl | 150-153 | 484.2 |
| 83 | 3-methoxyphenyl | 163-165 | 483.2 |
| 84 | 3-fluorophenyl | 165-168 | 471.2 |
| 85 | 3-chlorophenyl | 165-166 | 487.2 |
| 86 | 2,5-difluorophenyl | >150 dec | 489.2 |
| 87 | 3,5-difluorophenyl | >150 dec | 489.2 |

### EXAMPLE 88

### Evaluation of BACE-1 Binding Affinity of Test Compounds

Fluorescent Kinetic Assay

Final Assay Conditions: 10 nM human BACE1 (or 10 nM Murine BACEL), 25 μM substrate (WABC-6, MW 1549.6, from AnaSpec), Buffer: 50 mM Na-Acetate, pH 4.5, 0.05% CHAPS, 25% PBS, room temperature. Na-Acetate was from Aldrich, Cat.# 24, 124-5, was from Research Organics, Cat. # 1304C 1×, PBS was from Mediatech (Cellgro), Cat# 21-031-CV, peptide substrate AbzSEVNLDAEFRDpa was from AnaSpec, Peptide Name: WABC-6

Determination of stock substrate (AbzSEVNLDAEFRDpa) concentration: ˜25 mM stock solution is made in DMSO using the peptide weight and MW, and diluted to ˜25 μM (1:1000) in 1×PBS. Concentration is determined by absorbance at 354 nm using an extinction coefficient ε of 18172 M⁻ cm⁻¹, the concentration of stock substrate is corrected, and the substrate stock stored in small aliquots in −80° C. [Substrate Stock]=ABS 354 nm*10⁶/18172 (in mM) The extinction coefficient ε³⁵⁴ nm was adapted from TACE peptide substrate, which had the same quencher-fluorophore pair.

Determination of Stock Enzyme Concentration: the stock concentration of each enzyme is determined by absorbance at 280 nm using of 64150 M⁻¹ cm⁻¹ for hBACE1 and MuBACE1 in 6 M Guanidinium Hydrochloride (from Research Organics, Cat. # 5134G-2), pH˜6. The extinction coefficient ε280 nm for each enzyme was calculated based on known amino acid composition and published extinction coefficients for Trp (5.69 M⁻¹ cm⁻¹) and Tyr (1.28 M⁻¹ cm⁻¹) residues (Anal. Biochem. 182, 319-326).

Dilution and mixing steps: total reaction volume: 100 μL

2× inhibitor dilutions in buffer A(66.7 mM Na-Acetate, pH 4.5,0.0667% CHAPS) were prepared,

4× enzyme dilution in buffer A(66.7 mM Na-Acetate, pH 4.5,0.0667% CHAPS) were prepared,

100 μM substrate dilution in 1× PBS was prepared, and

50 μL 2× Inhibitor, 25 L 100 μM substrate are added to each well of 96-well plate (from DYNEX Technologies, VWR #: 11311-046), immediately followed by 25 μL 4× enzyme (added to the inhibitor and substrate mix), and the fluorescence readings are initiated.

Fluorescence Readings: Readings at Aex 320 nm and Aem 420 nm are taken every 40 sec for 30 min at room temperature and the linear slope for substrate cleavage rate (vᵢ) determined.

Calculation of % Inhibition:

% Inhibition=100*(1-vᵢ/v₀)
vᵢ: substrate cleavage rate in the presence of inhibitor
v₀: substrate cleavage rate in the absence of inhibitor
   IC₅₀ Determination:
% Inhibition=((B*IC₅₀ⁿ)+(100*I₀ⁿ))/(IC₅₀ⁿ+I₀ⁿ)

(Model # 39 from LSW Tool Bar Excel where B is the % inhibition from the enzyme control, which should be close to 0.) % Inhibition is plotted vs. Inhibitor Concentraition (I₀) and the data fit to the above equation to obtain IC₅₀ value and Hill number (n) for each compound. Testing at least 10 different inhibitor concentrations is preferred. The data shown in Table VI below.

TABLE VI

| Ex. No. | BACE1 (IC₅₀ μM) |
|---|---|
| 1 | C |
| 2 | A |
| 3 | A |
| 4 | A |
| 5 | A |
| 6 | B |
| 7 | A |
| 8 | C |
| 9 | C |
| 10 | A |
| 11 | B |
| 12 | A |
| 13 | A |
| 14 | B |
| 15 | A |
| 16 | A |
| 17 | B |
| 18 | C |
| 19 | A |
| 20 | A |
| 21 | A |
| 22 | A |
| 24 | A |
| 25 | A |
| 26 | C |
| 27 | C |
| 32 | C |
| 33 | A |
| 34 | A |
| 35 | A |
| 36 | A |
| 37 | A |
| 38 | A |
| 39 | A |
| 40 | A |
| 41 | A |
| 42 | A |
| 43 | B |
| 44 | A |
| 45 | A |
| 46 | A |
| 47 | C |
| 48 | C |
| 49 | A |
| 50 | A |
| 51 | A |
| 52 | A |
| 53 | A |
| 54 | A |
| 55 | A |
| 56 | B |
| 57 | B |
| 58 | A |
| 59 | C |
| 60 | C |
| 61 | C |
| 62 | C |
| 63 | C |
| 64 | C |
| 65 | C |
| 66 | C |
| 67 | C |
| 68 | C |
| 69 | C |
| 70 | C |
| 71 | C |
| 72 | C |
| 73 | C |
| 80 | A |
| 81 | A |
| 82 | A |
| 83 | A |
| 84 | A |
| 85 | A |
| 86 | A |
| 87 | A |

For Table VI
A = 0.01 μM-1.00 μM
B = 1.01 μM-3.00 μM
C = >3.00 μM

What is claimed is:

1. A compound of formula I 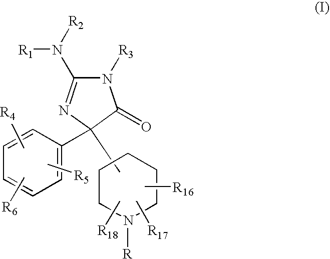
wherein
R is H, COR₇, CO₂R₇, CONR₈R₉, SO₂NR₈R₉, SO,RₘR₁₀, or an alkyl, alkenyl, alkynyl, cycloalkyl, cycloheteroalkyl, aryl or heteroaryl group each optionally substituted;
R₁, R₂, and R₃ are each independently H or an alkyl, cycloalkyl, cycloheteroalkyl, aryl or heteroaryl group each optionally substituted or R₁ and R₂ may be taken together with the atom to which they are attached form an optionally substituted 5- to 7-membered ring optionally interrupted by an additional heteroatom selected from O, N or S;
R₄, R₅, and R₆ are each independently H, halogen, NO₂, CN, OR₁₁, COR₁₁, CO₂R₁₁, CONR₁₂R₁₃, NR₁₂R₁₃, NR₁₂COR₁₄, NR₁₂SO₂R₁₄, SO₂NR₁₂R₁₃, SOₙR₄ or an alkyl, alkenyl, alkynyl, cycloalkyl, cycloheteroalkyl, aryl or heteroaryl group each optionally substituted or when attached to adjacent carbon atoms R₄ and R₅ or R₅ and R₆ may be taken together with the atoms to which they are attached to form an optionally substituted 5- to7-membered ring optionally interrupted by one, two or three heteroatoms selected from O, N or S;
m and n are each independently 0,1 or 2;
R₇ and R₁₁, are each independently H or an alkyl, alkenyl, alkynyl, cycloalkyl, cycloheteroalkyl, aryl or heteroaryl group each optionally substituted;
R₈, R₉, R₁₂ and R₁₃ are each independently H, OR₁₅, COR,₁₅, CO₂R₁₅ or an alkyl, alkenyl, alkynyl, cycloalkyl, cycloheteroalkyl, aryl or heteroaryl group each optionally substituted or R8 and R₉ or R₁₂ and R₁₃ may be taken together with the atom to which they are attached to form an optionally substituted 5- to 7-membered ring optionally interrupted by an additional he selected from O, N or S;
R₁₀ and R₁₄ are each independently an alkyl, alkenyl, alkynyl, cycloalkyl, cycloheteroalkyl, aryl or heteroaryl group each optionally substituted;
R₁₅ is H or an alkyl, alkenyl, alkynyl, cycloalkyl, cycloheteroalkyl, aryl or heteroaryl group each optionally substituted;
R₁₆, R₁₇ and R₁₈ are each independently H, halogen, CN, OR₁₉ or an alkyl, cycloalkyl, cyclohetereoalkyl, aryl or heteroaryl group each optionally substituted; and
R₁₉ is H or an alkyl, cycloalkyl, cyclohetereoalkyl, aryl or heteroaryl group each optionally substituted; or
a tautomer thereof, a stereoisomer thereof or a pharmaceutically acceptable salt thereof.

2. The compound according to claim 1 wherein R₁₆, R₁₇ and R₁₈ are H.

3. The compound according to claim 1 wherein R₁ and R₂ are H.

4. The compound according to claim 1 wherein R₃ is a C₁-C₄ alkyl group.

5. The compound according to claim 1 wherein R₆ is NR₁₂COR₁₄ or an optionally substituted aryl or heteroaryl group.

6. The compound according to claim 2 wherein the piperidinyl ring is attached in the 3- or 4-position.

7. The compound according to claim 2 wherein R₃ is C₁-C₄ alkyl and R₆ is NR₁₂COR₁₄ or an optionally substituted phenyl or heteroaryl group.

8. The compound according to claim 6 wherein R is COR₇ and R₁ and R₂ are H.

9. The compound according to claim 1 selected from the group consisting essentially of:
2-amino-5-(1,1′-biphenyl-3-yl)-5-(1-isobutyrylpiperidin-4-yl)-3-methyl-3,5-dihydro-4H -imidazol-4-one;
2-amino-5-(1-benzoylpiperidin-4-yl)-5-(1,1′-biphenyl-3-yl)-3-methyl-3,5-dihydro-4H -imidazol-4-one;
2-amino-5-(1,1′-biphenyl-3-yl)-5-[1-(3-methoxybenzoyl)piperidin-4-yl]-3-methyl-3,5-dihydro-4H-imidazol-4-one;
2-amino-5-(11′-biphenyl-3-yl)-5-[1-(2-furoyl)piperidin-4-yl]-3-methyl -3,5-dihydro-4H -imidazol-4-one;
2-amino-5-(1,1′-biphenyl-3-yl)-5-[1-(2-methoxybenzoyl)piperidin-4-yl]-3-methyl-3,5-dihydro-4H-imidazol-4-one;
2-amino-5-(1,1′-biphenyl-3-yl)-5-[1-(4-methoxybenzoyl)piperidin-4-yl]-3-methyl-3,5-dihydro-4H-imidazol-4-one;
2-amino-5-(1,1′-biphenyl-3-yl)-5-[1-(3,4-dimethoxybenzoyl)piperidin-4-yl]-3-methyl -3,5-dihydro-4H-imidazol-4-one;
2-amino-5-[1-(1,3-benzodioxol-5-ylcarbonyl)piperidin-4-yl]-5-(1,1′-biphenyl -3-yl)-3-methyl-3,5-dihydro-4H-imidazol-4-one;
2-amino-5-(1,1′-biphenyl-3-yl)-3-methyl-5-[1-(1-naphthoyl)piperidin-4-yl]-3,5-dihydro -4H-imidazol-4-one;
2-amino-5-(1,1′-biphenyl-3-yl)-3-methyl-5-[1-(4-propylbenzoyl)piperidin-4-yl]-3,5-dihydro-4H-imidazol-4-one;
2-amino-5-(1,1′-biphenyl-3-yl)-3-methyl-5-[1-(4-propoxybenzoyl)piperidin-4-yl]-3,5-dihydro-4H-imidazol-4-one;
2-({4-[2-amino4-(1,1′-biphenyl-3-yl)-1-methyl-5-oxo4,5-dihydro-1H -imidazol-4-yl]piperidin-1-yl)carbonyl)benzonitrile;
3-({4-[2-amino4-(1,1′-biphenyl-3-yl)-1-methyl-5-oxo-4,5-dihydro-1H -imidazol-4-yl]piperidin-1-yl}carbonyl)benzonitrile;
4-({4-[2-amino4-(1,1′-biphenyl-3-yl)-1-methyl-5-oxo4,5-dihydro-1H -imidazol-4-yl]piperidin-1-yl}carbonyl)benzonitrile;
2-amino-5-(1,1′-biphenyl-3-yl)-5-[1-(2-chloro-6-methylisonicotinoyl)piperidin-4-yl]-3-methyl-3,5-dihydro-4H-imidazol-4-one;
2-amino-5-(1,1′-biphenyl-3-yl)-5-[1-(3-furoyl)piperidin-4-yl]-3-methyl -3,5-dihydro-4H -imidazol-4-one;
2-amino-5-(1,1′-biphenyl-3-yl)-3-methyl-5-[1-(thien-2-ylcarbonyl)piperidin-4-yl]-3,5-dihydro-4H-imidazol-4-one;
2-amino-5-(1,1′-biphenyl-3-yl)-3-methyl-5-[1-(thien-3-ylcarbonyl)piperidin-4-yl]-3,5-dihydro-4H-imidazol-4-one;
2-amino-5-(1,1′-biphenyl-3-yl)-3-methyl-5-[1-(phenylsulfonyl)piperidin-4-yl]-3,5-dihydro-4H-imidazol-4-one;
2-amino-5-{1-[(benzyloxy)acetyl]piperidin-4-yl}-5-(1,1′-biphenyl-3-yl)-3-methyl-3,5-dihydro-4H-imidazol-4-one;
2-amino-5-(1,1′-biphenyl-3-yl)-3-methyl-5-(1-prop-2-ynylpiperidin-4-yl) -3,5-dihydro -4H-imidazol-4-one;
5-(1-acetylpiperidin-4-yl)-2-amino-5-(1,1′-biphenyl-3-yl)-3-methyl-3,5-dihydro -4H -imidazol-4-one;
2-amino-5-(1,1′-biphenyl-3-yl)-3-methyl-5-(1-propionylpiperidin-4-yl)-3,5-dihydro -4H -imidazol-4-one;
2-amino-5-(1,1′-biphenyl-3-yl)-5-(1-butyrylpiperidin-4-yl)-3-methyl-3,5-dihydro -4H -imidazol-4-one
2-amino-5-(1-benzoylpiperidin-4-yl)-5-(3-cyclohexylphenyl)-3-methyl-3,5-dihydro-4H -imidazol-4-one;
5-(1-acetylpiperidin-4-yl)-2-amino-5-(3-cyclohexylphenyl)-3-methyl-3,5-dihydro-4H -imidazol-4-one;
2-amino-5-(1-benzoylpiperidin-4-yl)-3-methyl-5-(3-pyridin-3-ylphenyl)-3,5-dihydro -4H -imidazol-4-one;
2-amino-5-(1-benzoylpiperidin-4-yl)-3-methyl-5-(3-pyrimidin-5-ylphenyl)-3,5-dihydro -4H-imidazol-4-one;
2-amino-5-(1-benzoylpiperidin-4-yl)-3-methyl-5-(3-pyrazin-2-ylphenyl)-3,5-dihydro -4H-imidazol-4-one;
2-amino-5-(1-benzoylpiperidin-4-yl)-5-(2′,5′-difluoro-1,1′-biphenyl-3-yl) -3-methyl-3,5-dihydro-4H-imidazol-4-one;
2-amino-5-(1-benzoylpiperidin-4-yl)-3-methyl-5-(3-propoxyphenyl)-3,5-dihydro-4H -imidazol-4-one;
2-amino-5-(1-benzoylpiperidin-4-yl)-5-(3-isobutoxyphenyl)-3-methyl-3,5-dihydro-4H -imidazol-4-one;
2-amino-5-(1-benzoylpiperidin-4-yl)-5-[3-(bui-3-ynyloxy)phenyl]-3-methyl-3,5-dihydro-4H-imidazol-4-one;
2-amino-5-(1-benzoylpiperidin-4-yl)-5-[3-(cyclopropylmethoxy)phenyl]-3-methyl-3,5-dihydro-4H-imidazol-4-one;
N-{3-[2-amino4-(1-benzoylpiperidin-4-yl)-1-methyl-5-oxo-4,5-dihydro-1H -imidazol-4-yl]phenyl}-2-methoxyacetamide;
3-[2-amino-4-(1-benzoylpiperidin-4-yl)-1-methyl-5-oxo-4,5-dihydro-1H -imidazol-4-yl]-N-isobutylbenzamide;
ethyl 3-(2-amino-1-methyl-5-oxo-4-phenyl-4,5-dihydro-1H-imidazol-4-yl)piperidine-1-carboxylate;
2-amino-5-[1-(2-furoyl)piperidin-3-yl]-3-methyl-5-phenyl-3,5-dihydro-4H -imidazol-4-one;
2-amino-5-[1-(isoxazol-5-yl)piperidin-3-yl]-3-methyl-5-phenyl-3,5-dihydro-4H -imidazol-4-one;
2-amino-3-methyl-5-phenyl-5-[1-(trifluoroacetyl)piperidin-3-yl]-3,5-d ihydro-4H -imidazol-4-one;
2-amino-5-[1-(cyclopentylcarbonyl)piperidin-3-yl]-3-methyl-5-phenyl-3,5-dihydro-4H -imidazol-4-one;
5-[1-(1-adamantylcarbonyl)piperidin-3-yl]-2-amino-3-methyl-5-phenyl-3,5-dihydro-4H -imidazol-4-one;
2-amino-5-(1-benzoylpiperidin-3-y)-3-methyl-5-phenyl-3,5-dihydro-4H-imidazol-4-one;
2-amino-3-methyl-5-phenyl-5-[1-(thien-2-ylcarbonyl)piperidin-3-yl]-3,5-dihydro-4H -imidazol-4-one;
2-amino-5-[1-(3-methoxybenzoyl)piperidin-3-yl]-3-methyl-5-phenyl-3,5-dihydro-4H -imidazol-4-one;
2-amino-3-methyl-5-[1-(3-methylbutanoyl)piperidin-3-yl]-5-phenyl-3,5-dihydro-4H -imidazol-4-one;
4-[3-(2-amino-1-methyl-5-oxo-4-phenyl-4,5-dihydro-1H-imidazol-4-yl)piperidin-1-yl]-4-oxobutanoic acid;
{2-[3-(2-amino-l-methyl-5-oxo-4-phenyl-4,5-dihydro-1H-imidazol-4-yl)piperidin-1-yl]-2-oxoethoxy}acetic acid;
5-[3-(2-amino-1-methyl-5-oxo-4-phenyl-4,5-dihydro-1H-imidazol-4-yl)piperidin-1-yl]-5-oxopentanoic acid;
2-amino-5-(1-benzoylpiperidin-4-yl)-5-[3-(2-fluoropyridin-3-yl)phenyl]-3-methyl-3,5-dihydro-4H-imidazol-4-one;
2-amino-5-(1-benzoylpiperidin-4-yl)-3-methyl-5-(3-pyrimidin-5-ylphenyl)-3,5-dihydro -4H-imidazol-4-one;
2-amino-5-(1-benzoylpiperidin-4-y5)-5-[3-(5-methoxypyridin-3-yl)phenyl]-3-methyl-3,5-dihydro-4H-imidazol-4-one;
2-amino-5-(1-benzoylpiperidin-4-yl)-5-(3′-methoxybiphenyl-3-yl)-3-methyl-3,5-dihydro-4H-imidazol-4-one;
2-amino-5-(1-benzoylpiperidin-4-yl)-5-(3′-fluorobiphenyl-3-yl)-3-methyl-3,5-dihydro -4H-imidazol-4-one;
2-amino-5-(1-benzoylpiperidin-4-yl)-5-(3′-chlorobiphenyl-3-yl)-3-methyl-3,5-dihydro -4H-imidazol-4-one;
2-amino-5-(1-benzoylpiperidin-4-yl)-5-(2′,5′-difluorobiphenyl-3-yl)-3-methyl-3,5-dihydro-4H-imidazol-4-one;
2-amino-5-(1-benzoylpiperidin-4-yl)-5-(3′,5′-difluorobiphenyl-3-yl)-3-methyl-3,5-dihydro-4H-imidazol-4-one;
a tautomer thereof;
a stereoisomer thereof; and
a pharmaceutically acceptable salt thereof.

10. A method for the treatment of a disease or disorder associated with excessive BACE activity in a patient in need thereof which comprises providing to said patient a therapeutically effective amount of a compound of formula I 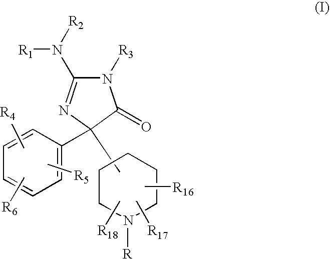
wherein
R is H, COR₇, CO₂R₇, CONR₈R₉, SO₂NR₈R₉, SOₘR₁₀, or an alkyl, alkenyl, alkynyl, cycloalkyl, cycloheteroalkyl, aryl or heteroaryl group each optionally substituted;
R₁, R₂, and R₃ are each independently H or an alkyl, cycloalkyl, cycloheteroalkyl, aryl or heteroaryl group each optionally substituted or R₁ and R₂ may be taken together with the atom to which they are attached form an optionally substituted 5- to 7-membered ring optionally interrupted by an additional heteroatom selected from O, N or S;
R₄, R₅, and R₆ are each independently H, halogen, NO₂, CN, OR₁₁, COR₁₁, CO₂R₁₁, CONR₁₂R₁₃, NR₁₂R₁₃, NR₁₂COR₁₄, NR₁₂SO₂R₁₄, S0₂NR₁₂R₁₃, SOₙR₁₄ or an alkyl, alkenyl, alkynyl, cycloalkyl, cycloheteroalkyl, aryl or heteroaryl group each optionally substituted or when attached to adjacent carbon atoms R₄ and R₅ or R₅ and R₆ may be taken together with the atoms to which they are attached to form an optionally substituted 5- to7-membered ring optionally interrupted by one, two or three heteroatoms selected from O, N or S;
m and n are each independently 0,1 or 2;
R₇ and R₁₁ are each independently H or an alkyl, alkenyl, alkynyl, cycloalkyl, cycloheteroalkyl, aryl or heteroaryl group each optionally substituted;
R₈, R₉, R₁₂ and R₁₃ are each independently H, OR₁₅, COR₁₅, CO₂R₁₅ or an alkyl, alkenyl, alkynyl, cycloalkyl, cycloheteroalkyl, aryl or heteroaryl group each optionally substituted or R₈ and R₉ or R₁₂ and R₁₃ may be taken together with the atom to which they are attached to form an optionally substituted 5- to 7-membered ring optionally interrupted by an additional heteroatom selected from O, N or S;
R₁₀ and R₁₄ are each independently an alkyl, alkenyl, alkynyl, cycloalkyl, cycloheteroalkyl, aryl or heteroaryl group each optionally substituted;
R₁₅ is H or an alkyl, alkenyl, alkynyl, cycloalkyl, cycloheteroalkyl, aryl or heteroaryl group each optionally substituted;
R₁₆, R₁₇ and R₁₈ are each independently H, halogen, CN, OR₁₉ or an alkyl, cycloalkyl, cyclohetereoalkyl, aryl or heteroaryl group each optionally substituted; and
R₁₉ is H or an alkyl, cycloalkyl, cyclohetereoalkyl, aryl or heteroaryl group each optionally substituted; or
a tautomer thereof, a stereoisomer thereof or a pharmaceutically acceptable salt thereof.

11. The method according to claim 10 wherein said disease or disorder is selected from the group consisting essentially of: Alzheimer's disease; cognitive impairment; Down's Syndrome; HCHWA-D; cognitive decline; senile dementia; cerebral amyloid angiopathy; and a neurodegenerative disorder.

12. The method according to claim 10 wherein said disease or disorder is characterized by the production of β-amyloid deposits or neurofibrillary tangles.

13. The method according to claim 11 wherein said disease or disorder is Alzheimer's disease.

14. A method for modulating the activity of BACE which comprises contacting a receptor thereof with an effective amount of a compound according to claim 1.

15. A pharmaceutical composition which comprises a pharmaceutically acceptable carrier and an effective amount of a compound of formula I 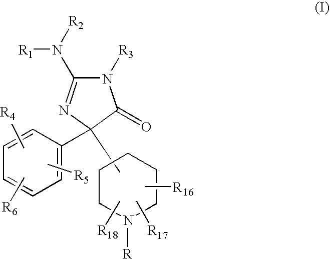
wherein
R is H, COR₇, CO₂R₇, CONR₈R₉, SO₂NR₈R₉, SOₘR₁₀, or an alkyl, alkenyl, alkynyl, cycloalkyl, cycloheteroalkyl, aryl or heteroaryl group each optionally substituted;
R₁, R₂, and R₃ are each independently H or an alkyl, cycloalkyl, cycloheteroalkyl, aryl or heteroaryl group each optionally substituted or R₁ and R₂ may be taken together with the atom to which they are attached form an optionally substituted 5- to 7-membered ring optionally interrupted by an additional heteroatom selected from O, N or S;
R₄, R₅, and R₆ are each independently H, halogen, NO₂, CN, OR₁₁, COR₁₁, CO₂R₁₁, CONR₁₂R₁₃, NR₁₂R₁₃, NR₁₂COR₁₄, NR₁₂SO₂R₁₄, SO₂NR₁₂R₁₃, SOₙR₁₄ or an alkyl, alkenyl, alkynyl, cycloalkyl, cycloheteroalkyl, aryl or heteroaryl group each optionally substituted or when attached to adjacent carbon atoms R₄ and R₅ or R₅ and R₆ may be taken together with the atoms to which they are attached to form an optionally substituted 5- to7-membered ring optionally interrupted by one, two or three heteroatoms selected from O, N or S;
m and n are each independently 0,1 or 2;
R₇ and R₁₁ are each independently H or an alkyl, alkenyl, alkynyl, cycloalkyl, cycloheteroalkyl, aryl or heteroaryl group each optionally substituted;
R₈, R₉, R₁₂ and R₁₃ are each independently H, OR₁₅, COR₁₅, CO₂R₁₅ or an alkyl, alkenyl, alkynyl, cycloalkyl, cycloheteroalkyl, aryl or heteroaryl group each optionally substituted or R₈ and R₉ or R₁₂ and R₁₃ may be taken together with the atom to which they are attached to form an optionally substituted 5- to 7-membered ring optionally interrupted by an additional heteroatom selected from O, N or S;
R₁₀ and R₁₄ are each independently an alkyl, alkenyl, alkynyl, cycloalkyl, cycloheteroalkyl, aryl or heteroaryl group each optionally substituted;
R₁₅ is H or an alkyl, alkenyl, alkynyl, cycloalkyl, cycloheteroalkyl, aryl or heteroaryl group each optionally substituted;
R₁₆, R₁₇ and R₁₈ are each independently H, halogen, CN, OR₁₉ or an alkyl, cycloalkyl, cyclohetereoalkyl, aryl or heteroaryl group each optionally substituted; and
R₁₉ is H or an alkyl, cycloalkyl, cyclohetereoalkyl, aryl or heteroaryl group each optionally substituted; or
a tautomer thereof, a stereoisomer thereof or a pharmaceutically acceptable salt thereof.

16. The composition according to claim 15 having a formula I compound wherein R₁₆, R₁₇ and R18 are H.

17. The composition according to claim 16 having a formula I compound wherein R₁ and R₂ are H.

18. The composition according to claim 17 having a formula I compound wherein R₃ is methyl.

19. The composition according to claim 18 having a formula I compound wherein the piperidinyl ring is attached in the 3- or 4-position; R is COR₇; and R₆ is NR₁₂COR₁₄ or an optionally substituted phenyl or heteroaryl group.

20. The composition according to claim 15 having a formula I compound selected from the group consisting essentially of:
2-amino-5-(1,1′-biphenyl-3-yl)-5-(1-isobutyrylpiperidin-4-yl)-3-methyl-3,5-dihydro-4H -imidazol-4-one;
2-amino-5-(1-benzoylpiperidin-4-yl)-5-(1,1′-biphenyl-3-yl)-3-methyl-3,5-dihydro-4H -imidazol-4-one;
2-amino-5-(1,1′-biphenyl-3-yl)-5-[1-(3-methoxybenzoyl)piperidin-4-yl]-3-methyl-3,5-dihydro-4H-imidazol-4-one;
2-amino-5-(1,1′-biphenyl-3-yl)-5-[1-(2-furoyl)piperidin-4-yl]-3-methyl -3,5-dihydro-4H -imidazol-4-one;
2-amino-5-(1,1′-biphenyl-3-yl)-5-[1-(2-methoxybenzoyl)piperidin-4-yl]-3-methyl-3,5-dihydro-4H-imidazol-4-one;
2-amino-5-(1,1′-biphenyl-3-yl)-5-[1-(4-methoxybenzoyl)piperidin-4-yl]-3-methyl-3,5-dihydro-4H-imidazol-4-one;
2-amino-5-(1,1′-biphenyl-3-yl)-5-[1-(3,4-dimethoxybenzoyl)piperidin-4-yl]-3-methyl -3,5-dihydro-4H-imidazol-4-one;
2-amino-5-[1-(1,3-benzodioxol-5-ylcarbonyl)piperidin-4-yl]-5-(1,1′-biphenyl-3-yl)-3-methyl-3,5-dihydro-4H-imidazol-4-one;
2-amino-5-(1,1′-biphenyl-3-yl)-3-methyl-5-[1-(1-naphthoyl)piperidin-4-yl]-3,5-dihydro -4H-imidazol-4-one;
2-amino-5-(1,1′-biphenyl-3-yl)-3-methyl-5-[1-(4-propylbenzoyl)piperidin-4-yl]-3,5-dihydro-4H-imidazol-4-one;
2-amino-5-(1,1′-biphenyl-3-yl)-3-methyl-5-[1-(4-propoxybenzoyl)piperidin-4-yl]-3,5-dihydro-4H-imidazol-4-one;
2-({4-[2-amino-4-(1,1′-biphenyl-3-yl)-1-methyl-5-oxo-4,5-dihydro-1H -imidazol-4-yl]piperidin-1-yl}carbonyl)benzonitrile;
3-({4-[2-amino4-(, 1′-biphenyl-3-yl)-1-methyl-5-oxo-4,5-dihydro-1H -imidazol-4-yl]piperidin-1-yl}carbonyl)benzonitrile;
4-({4-[2-amino-4-(1,1′-biphenyl-3-yl)-1-methyl-5-oxo-4,5-dihydro-1H -imidazol-4-yl]piperidin-1-yl}carbonyl)benzonitrile;
2-amino-5-(1,1′-biphenyl-3-yl)-5-[1-(2-chloro-6-methylisonicotinoyl)piperidin -4-yl]-3-methyl-3,5-dihydro-4H-imidazol-4-one;
2-amino-5-(1,1′-biphenyl-3-yl)-5-[1-(3-furoyl)piperidin-4-yl]-3-methyl -3,5-dihydro-4H -imidazol-4-one;
2-amino-5-(1,1′-biphenyl-3-yl)-3-methyl-5-[1-(thien-2-ylcarbonyl)piperidin-4-yl]-3,5-dihydro-4H-imidazol-4-one;
2-amino-5-(1,1′-biphenyl-3-yl)-3-methyl-5-[1-(thien-3-ylcarbonyl)piperidin-4-yl]-3,5-dihydro-4H-imidazol-4-one;
2-amino-5-(1,1′-biphenyl-3-yl)-3-methyl-5-[1-(phenylsulfonyl)piperidin-4-yl]-3,5-dihydro-4H-imidazol-4-one;
2-amino-541-[(benzyloxy)acetyl]piperidin-4-yl}-5-(1,1′-biphenyl-3-yl)-3-methyl -3,5-dihydro-4H-imidazol-4-one;
2-amino-5-(1,1′-biphenyl-3-yl)-3-methyl-5-(1-prop-2-ynylpiperidin-4-yl) -3,5-dihydro -4H-imidazol-4-one;
5-(1-acetylpiperidin-4-yl)-2-amino-5-(1,1′-biphenyl-3-yl)-3-methyl-3,5-dihydro-4H -imidazol-4-one;
2-amino-5-(, 1′-biphenyl-3-yl)-3-methyl-5-(1-propionylpiperidin-4-yl)-3,5-dihydro-4H -imidazol-4-one;
2-amino-5-(1,1′-biphenyl-3-yl)-5-(1-butyrylpiperidin-4-yl)-3-methyl-3,5-dihydro-4H -imidazol-4-one 2-amino-5-(1-benzoylpiperidin-4-yl)-5-(3-cyclohexylphenyl)-3-methyl-3,5-dihydro-4H -imidazol-4-one;
5-(1-acetylpiperidin-4-yl)-2-amino-5-(3-cyclohexylphenyl)-3-methyl-3,5-dihydro-4H -imidazol-4-one;
2-amino-5-(1-benzoylpiperidin-4-yl)-3-methyl-5-(3-pyridin-3-ylphenyl)-3,5-dihydro-4H -imidazol-4-one;
2-amino-5-(1-benzoylpiperidin-4-y)-3-methyl-5-(3-pyrimidin-5-ylphenyl)-3,5-dihydro -4H-imidazol-4-one;
2-amino-5-(1-benzoylpiperidin-4-yl)-3-methyl-5-(3-pyrazin-2-yphenyl)-3,5-dihydro -4H-imidazol-4-one;
2-amino-5-(1-benzoylpiperidin-4-yl)-5-(2′,5′-difluoro-1,1′-biphenyl-3-yl)-3-methyl-3,5-dihydro-4H-imidazol-4-one;
2-amino-5-(1-benzoylpiperidin-4-yl)-3-methyl-5-(3-propoxyphenyl)-3,5-dihydro-4H -imidazol-4-one;
2-amino-5-(1-benzoylpiperidin-4-yl)-5-(3-isobutoxyphenyl)-3-methyl-3,5-dihydro-4H -imidazol-4-one;
2-amino-5-(1-benzoylpiperidin-4-yl)-5-[3-(but-3-ynyloxy)phenyl]-3-methyl-3,5-dihydro -4H-imidazol-4-one;
2-amino-5-(1-benzoylpiperidin-4-yl)-5-[3-(cyclopropylmethoxy)phenyl]-3-methyl-3,5-dihydro-4H-imidazol-4-one;
N-{3-[2-amino4-(1-benzoylpiperidin-4-yl)-1-methyl-5-oxo4,5-dihydro-1H-imidazol 4-yl]phenyl}-2-methoxyacetamide;
3-[2-amino-4-(1-benzoylpiperidin-4-yl)-1-methyl-5-oxo4,5-dihydro-1H-imidazol-4-yl]-N-isobutylbenzamide;
ethyl 3-(2-amino-1-methyl-5-oxo-4-phenyl4,5-dihydro-1H-imidazol-4-yl)piperidine-1-carboxylate;
2-amino-5-[1-(2-furoyl)piperidin-3-yl]-3-methyl-5-phenyl-3,5-dihydro-4H -imidazol-4-one;
2-amino-5-[1-(isoxazol-5-yl)piperidin-3-yl]-3-methyl-5-phenyl-3,5-dihydro-4H -imidazol-4-one;
2-amino-3-methyl-5-phenyl-5-[1-(trifluoroacetyl)piperidin-3-yl]-3,5-dihydro-4H -imidazol-4-one;
2-amino-5-[1-(cyclopentylcarbonyl)piperidin-3-yl]-3-methyl-5-phenyl-3,5-dihydro-4H -imidazol-4-one;
5-[1-(1-adamantylcarbonyl)piperidin-3-yl]-2-amino-3-methyl-5-phenyl-3,5-dihydro -4H -imidazol-4-one;
2-amino-5-(1-benzoylpiperidin-3-yl)-3-methyl-5-phenyl-3,5-dihydro-4H-imidazol-4-one;
2-amino-3-methyl-5-phenyl-5-[1-(thien-2-ylcarbonyl)piperidin-3-yl]-3,5-dihydro -4H -imidazol-4-one;
2-amino-5-[1-(3-methoxybenzoyl)piperidin-3-yl]-3-methyl-5-phenyl-3,5-dihydro-4H -imidazol-4-one;
2-amino-3-methyl-5-[1-(3-methylbutanoyl)piperidin-3-yl]-5-phenyl-3,5-dihydro -4H -imidazol-4-one;
4-[3-(2-amino-1-methyl-5-oxo-4-phenyl4,5-dihydro-1H-imidazol-4-yl)piperidin -1-yl]4-oxobutanoic acid;
{2-[3-(2-amino-1-methyl-5-oxo4-phenyl-4,5-dihydro-1H-imidazol-4-yl)piperidin -1-yl]-2-oxoethoxy}acetic acid;
5-[3-(2-amino-1-methyl-5-oxo-4-phenyl4,5-dihydro-1H-imidazol-4-yl)piperidin -1-yl]-5-oxopentanoic acid;
2-amino-5-(1-benzoylpiperidin-4-yl)-5-[3-(2-fluoropyridin-3-yl)phenyl]-3-methyl -3,5-dihydro-4H-imidazol-4-one;
2-amino-5-(1-benzoylpiperidin-4-yl)-3-methyl-5-(3-pyrimidin-5-ylphenyl)-3,5-dihydro -4H-imidazol-4-one;
2-amino-5-(1-benzoylpiperidin-4-yl)-5-[3-(5-methoxypyridin-3-yl)phenyl]-3-methyl-3,5-dihydro-4H-imidazol-4-one;
2-amino-5-(1-benzoylpiperidin-4-yl)-5-(3′-methoxybiphenyl-3-yl)-3-methyl-3,5-dihydro-4H-imidazol-4-one;
2-amino-5-(1-benzoylpiperidin-4-yl)-5-(3′-fluorobiphenyl-3-yl)-3-methyl-3,5-dihydro -4H-imidazol-4-one;
2-amino-5-(1-benzoylpiperidin-4-yl)-5-(3′-chlorobiphenyl-3-yl)-3-methyl-3,5-dihydro -4H-imidazol-4-one;
2-amino-5-(1-benzoylpiperidin-4-yl)-5-(2′,5′-difluorobiphenyl-3-yl)-3-methyl-3,5-dihydro-4H-imidazol-4-one;
2-amino-5-(1-benzoylpiperidin-4-yl)-5-(3′,5′-difluorobiphenyl-3-y)-3-methyl-3,5-dihydro-4H-imidazol-4-one;
a tautomer thereof;
a stereoisomer thereof; and
a pharmaceutically acceptable salt thereof.
